(19) United States
(12) Patent Application Publication
(10) Pub. No.: US 2008/0194545 A1
(43) Pub. Date: Aug. 14, 2008

(54) ANTIMICROBIAL COMPOSITIONS AND METHODS OF USE

(75) Inventors: Xiaoming Li, San Diego, CA (US); John M. Finn, San Diego, CA (US); Mark T. Hilgers, San Diego, CA (US)

Correspondence Address: DLA PIPER US LLP, 4365 EXECUTIVE DRIVE, SUITE 1100, SAN DIEGO, CA, 92121-2133, US

(73) Assignee: TRIUS THERAPEUTICS, San Diego, CA (US)

(21) Appl. No.: 12/027,950

(22) Filed: Feb. 7, 2008

Related U.S. Application Data
(60) Provisional application No. 60/900,489, filed on Feb. 8, 2007.

(51) Int. Cl. A61K 31/551; C07D 403/12; C07D 403/14; A61K 31/541; A61K 31/506; A61K 31/53; A61P 31/04; A61K 31/5377; C07D 417/14; C07D 413/14

(52) U.S. Cl. 514/218; 544/321; 544/295; 544/296; 540/575; 544/62; 544/123; 544/212; 514/228.2; 514/234.5; 514/252.19; 514/272; 514/245

(57) ABSTRACT

The present invention is directed to compounds of formula I, pharmaceutical compositions comprising the compounds, and methods for making and using the inventive compounds.

### CROSS-REFERENCE TO RELATED APPLICATIONS

The present application claims benefit under 35 USC §119(e) of provisional application 60/900,489, filed Feb. 8, 2007, which is hereby incorporated by reference in its entirety.

### FIELD OF THE INVENTION

The present invention relates generally to compositions and methods for preventing and/or treating bacterial infections and more specifically to compounds and pharmaceutical compositions that inhibit bacterial methionyl tRNA synthetase enzymes and methods of use as antimicrobial agents.

### BACKGROUND INFORMATION

Treatment of bacterial infections necessitates a continuous supply of new drugs to overcome drug resistance. The discovery and development of pseudomonic acid, which inhibits bacterial isoleucinyl-tRNA synthetase, has validated amino acyl tRNA synthetases as essential bacterial targets.

### SUMMARY OF THE INVENTION

In one embodiment, the invention provides anti-bacterial pyrimidine-based compounds

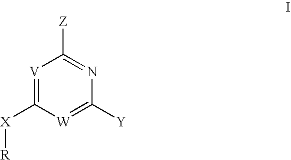

where X is O, S, N, or C,
exactly one of V and W is N, and the other is C—R1,
R is optionally substituted aryl or heteroaryl,
R1 is H or lower alkyl,
Y is optionally substituted 1H-benzimidazol-2-ylmethyl, 1H-imidazol-2-ylmethyl or 2-(4-oxo-4,4a-dihydroquinolin-2-yl)hydrazine and
Z is an optionally substituted amino, thio, or heterocyclyl group,
as well as diastereomers, enantiomers or a pharmaceutically acceptable salt, solvates, esters and prodrugs associated with these compounds, and methods of making and using them.

The invention provides a method of making a compound of claim 1, comprising the step of treating a compound of formula I

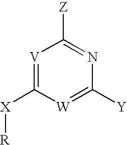

where
X═O,
R is optionally substituted phenyl or quinol-6-yl,
exactly one of V and W is N, and the other is CH,
and Y and Z are both selected from the group consisting of halo, alkylthio, alkylsulfonate, alkylsulfinate and optionally substituted 1H-benzimidazol-2-ylmethyl.

In another embodiment, the invention provides a method for treating a bacterial infection in a patient, comprising administering to the patient an effective amount of a pharmaceutical composition as described herein. In one aspect, the bacterium is Gram-positive, for example, Staphylococcus, Streptococcus, Enterococcus, Clostridium, Haemophilus, or Listeria spp. In a particular aspect, the bacterium is Staphylococcus aureus.

In one embodiment, a compound of the invention is administered at a dosage between about 1 and 1000 mg/kg.; between about 100 and 1000 mg/kg; or about 10 and 100 mg/kg.

Routes of administration include intravenous, oral, rectal, intramuscular, subcutaneous, and pulmonary administration, for example.

### DETAILED DESCRIPTION OF THE INVENTION

### Definitions

Unless otherwise specified, technical terms used have the meanings specified in the McGraw-Hill Dictionary of Scientific and Technical Terms, 6^{th} edition. All patents and publications referred to herein are incorporated by reference in their entirety.

“Heterocyclyl” refers to 5, 6 or 7 atom aromatic and non-aromatic heterocycles comprising at least one N, S, or O atom, the heterocycle optionally substituted with up to four different substituents, as well as bicyclic and tricyclic heterocycles optionally substituted with up to four different substituents and attached at any suitable position.

“Heteroaryl” refers to an aromatic heterocyclyl group.

“Lower alkyl” refers to C₁₋₆ saturated linear, branched, or cyclic alkyl groups.

“Prodrug” refers to a compound that, upon in vivo administration undergoes biological transformation to a pharmaceutically active compound. To produce a prodrug, those of skill in the art modify pharmaceutically active compound such that metabolic processes will regenerate the active compound (see, e.q., Nogrady (1985) Medicinal Chemistry A Biochemical Approach, Oxford University Press, New York, pages 388-392).

The present invention provides compounds having the structure

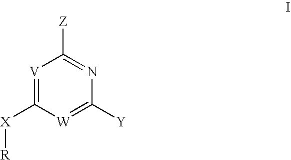

where
where X is O, S, N, or C,
exactly one of V and W is N, and the other is C—R1,
R is optionally substituted aryl or heteroaryl,
R1 is H or lower alkyl,
Y is optionally substituted 1H-benzimidazol-2-ylmethyl, 1H-imidazol-2-ylmethyl or 2-(4-oxo-4,4a-dihydroquinolin-2-yl)hydrazine and
Z is an optionally substituted amino, thio, or heterocyclyl group,
and diastereomers, enantiomers, and pharmaceutically acceptable salt, solvates, esters and prodrugs thereof.
Preferably X═O or S, with O especially preferred.

Preferably, R is a quinoline or a phenyl group bearing at least one substituent selected from the group consisting of halo, C₁₋₆ alkyl, C₁₋₆ alkoxy, trifluoromethyl, trifluoromethoxy, methylenedioxy, and cyano.

More preferably R is an 5-chloro-quinol-6-yl, 5-chloro-8-methoxy-quinol-6-yl 2,4-dichlorophenyl, 2-chloro-4-methoxyphenyl, 3,4-dimethoxyphenyl, 2-chloro-4-fluorophenyl, 2-methyl-4-fluorophenyl, 2,4-difluorophenyl, 4-trifluoromethoxyphenyl, 3-methyl-4-chlorophenyl, 3,4-methylenedioxphenyl, 4-methoxyphenyl, 2-chloro-4-cyanophenyl, 2-chloro-4-trifluoromethylphenyl, 3,4,5-trimethoxyphenyl, 2,4-dichloro-3-methylphenyl, and 2-chloro4,5-dimethoxyphenyl group.

Most preferably, R is 2,4-dichlorophenyl, 2-chloro-4-methoxyphenyl, or 2-chloro4,5-dimethoxyphenyl.

Preferably R₁ is H.

Preferably Z is H, NH₂, —SMe, —S(O)₂—, a saturated, partially saturated or unsaturated 5-7-membered monocyclic or 6-11-membered bicyclic ring containing 1-4 atoms selected from N, O, and S, where the available carbon atoms of the ring are substituted by 0, 1 or 2 oxo or thioxo groups.

More preferably, Z is NRₐR_{b}, where Rₐ and R_{b} are independently selected from the group consisting of H and C₁₋₄ alkyl and cycloalkyl, and the optionally substituted heterocyclic groups morphin-1-yl, thiomorphin-1-yl, piperazin-1-yl, pyrrolidin-1-yl, imidazol-1-yl, piperidin-1-yl, and 1,4-diazepan-1-yl.

Still more preferably, Z is optionally substituted morpholin-4-yl, dimethylamino, 4-acetylpiperazin-1-yl, 3,5-dimethylpiperazin-1-yl, 4-[2-(dimethylamino)ethyl]piperazin-1-yl, 3-hydroxypyrrolidin-1-yl, 3-oxopiperazin-1-yl, 4-(furan-2-ylcarbonyl)piperazin-1-yl, 4-methyl-1H-imidazol-1-yl, 4-morpholin-4-ylpiperazin-1-yl, 4-acetyl-1,4-diazepan-1-yl, 4-pyrimidin-2-ylpiperazin-1-yl, 4-(cyclopropylcarbonyl)piperazin-1-yl, 1,1-dioxidothiomorpholin-4-yl, 4-pyridin-2-ylpiperazin-1-yl, 3-[acetyl(ethyl)amino]pyrrolidin-1-yl, 4-(2-methylpropanoyl)piperazin-1-yl, 4-[5-(trifluoromethyl)pyridin-2-yl]piperazin-1-yl, 4-pyrazin-2-ylpiperazin-1-yl, 4-(2-morpholin-4-ylethyl)piperazin-1-yl, 4-pyridin-4-ylpiperazin-1-yl, 4-(4-methoxypyrimidin-2-yl)piperazin-1-yl, 3-oxo-1,4-diazepan-1-yl, 4-(1,3,5-triazin-2-yl)piperazin-1-yl, 4-(1,3-thiazol-2-yl)piperazin-1-yl, 4-[2-(1H-imidazol-1-yl)ethyl]piperazin-1-yl, 4-[(1,3-thiazol-2-ylamino)acetyl]piperazin-1-yl, 4-(morpholin-4-ylcarbonyl)piperazin-1-yl, 4-(pyrrolidin-1-ylcarbonyl)piperazin-1-yl, 4-[2-(2-methyl-1H-imidazol-1-yl)ethyl]piperazin-1-yl, 4-(2-pyrrolidin-1-ylethyl)piperazin-1-yl, or 4-[(2-oxopyrrolidin-1-yl)methyl]piperidin-1-yl.

Most preferably, Z is optionally substituted 4-acetylpiperazin-1-yl, 3-oxopiperazin-1-yl, dimethylamino, 4-pyrimidin-2-ylpiperazin-1-yl, 3,5-dimethylpiperazin-1-yl, 4-(cyclopropylcarbonyl)piperazin-1-yl, 4-pyridin-2-ylpiperazin-1-yl, 4-(2-methylpropanoyl)piperazin-1-yl, 4-(2-morpholin-4-ylethyl)piperazin-1-yl.

Examples of suitable substituents for phenyl and heterocyclic groups include C₁₋₆ alkyl, C₁₋₆ alkenyl, C₁₋₆ alkynyl, halogen, OH, CN, amino, C₁₋₆ alkylamino, C₁₋₆ dialkylamino, C₁₋₆ aminoalkyl, mercapto, methylthio, methylsulfinyl, methylsulfonyl, nitro, C₁₋₆ alkoxy, C₁₋₆alkyloxyalkyl, acyloxy, acylamino, carboxylic acid, carboxaldehyde, C₁₋₆ hydroxyalkyl, carboxyamino, alkoxycarbonyl, carboxamide, aryl and heteroaryl.

### Methods of Preparation

The compounds of the present invention may be prepared through use of chemical synthetic methods well known to those of skill in the art. Any known method, including those specifically exemplified herein, may be used to synthesize compounds of the present invention.

### Reaction Schemes

The following reaction schemes illustrate the synthesis of compounds and the variety of reactions that may be used to prepare the intermediates from which compounds of formula I may be prepared.

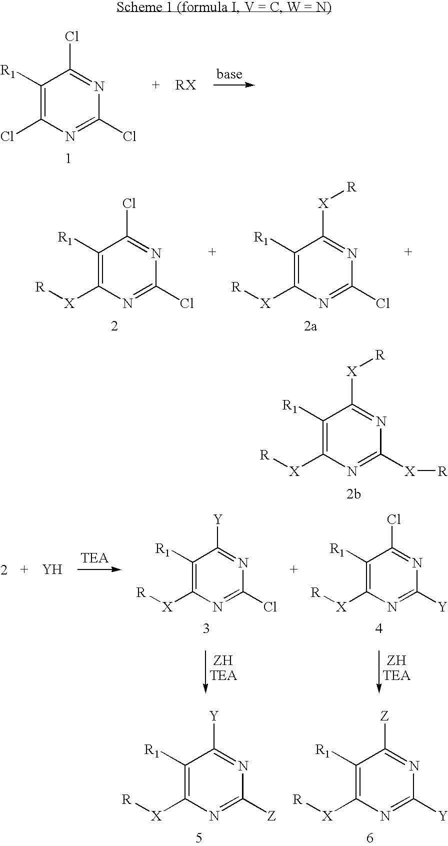

Scheme I illustrates a general method for forming compounds where V═C, W═N, X═O or S, and R is aryl or heteroaryl. Compound 2 can be formed by reaction of compound 1 with a nucleophile RX, such as 2-chloro-4-methoxyphenol, in the presence of a base e.g. an alkali metal alkoxide, hydroxide, or carbonate in either a protic or aprotic solvent (e.g. toluene, THF, ETOH, iso-propanol, CH₃CN, or DMF) at 78° C. to 120° C. (step 1). Compound 2 from step 1 can subsequently be treated with YH in the presence of Et₃N, di-isopropylethylamine (DIEA) or an alkali metal carbonate in the same protic or aprotic solvents to produce compounds 3 and 4 (step 2). Compounds (3 and 4) from step 2 can be further treated with ZH (free base or HCl salt form) in the presence of Et₃N, DIEA or an alkali metal carbonate to yield the target compounds (5 and 6), respectively (step 3). Step 3 can also been conducted neat where ZH is dimethyl amine, diethyl amine or dipropyl amine.

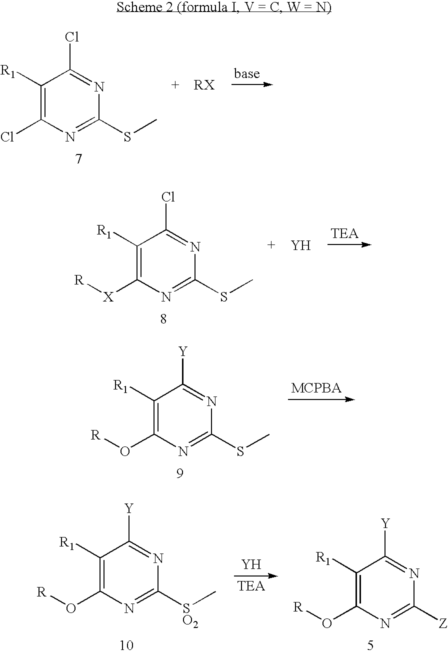

Scheme 2 illustrates an alternative method for forming a compound of formula I with V═C and W═N (isomer 5). Compound 8 can be formed by treating compound 7 with RX such as 2-chloro-4-methoxyphenol, in the presence of an organic or inorganic base e.g. alkalimetal alkoxide, hydroxide, carbonate (step 1). Compound 8 from step 1 can subsequently be treated with YH in the presence of Et₃N, DIEA or an alkali metal carbonate to produce compound 9 (step 2). Compound 9 from step 2 can be treated with excess meta-chloroperbenzoic acid (MCPBA) in the presence of CH₂Cl₂, CHCl₃, or THF at 23° C. to 78° C. to produce compound 10 (step 3). Compound 10 can further be treated with ZH in the presence of Et₃N, DIEA or an alkali metal carbonate to afford target compounds 5 (step 4). Step 4 can also been conducted neat where ZH is dimethyl amine, diethyl amine or dipropyl amine.

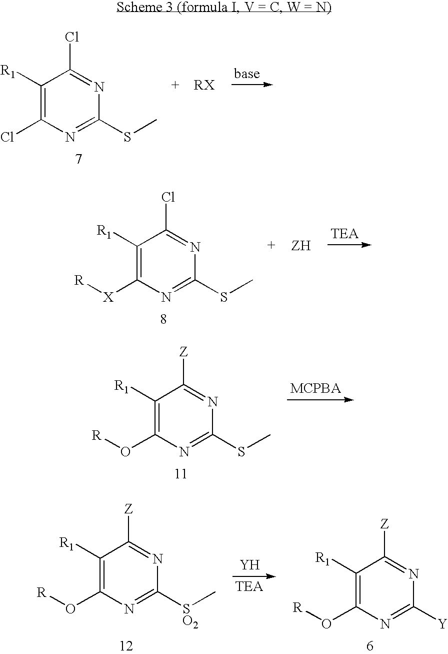

Scheme 3 illustrates a method similar to Scheme 2 for forming compound of formula I with V═N and W═C (isomer 6). Compound 8 from step 1 of Scheme 2 can be treated with ZH in the presence of Et₃N, DIEA or an alkali metal carbonate to produce compound 11 (step 2). Compound 11 from step 2 can be treated with excess MCPBA in the presence of CH₂Cl₂, CHCl₃, or THF at 23° C. to 78° C. to produce compound 12 (step 3). Compound 12 can further be treated with ZH in the presence of Et₃N, DIEA or an alkali metal carbonate to produce compound 6 (step 4). Step 4 can also been conducted neat where ZH is dimethyl amine, diethyl amine or dipropyl amine.

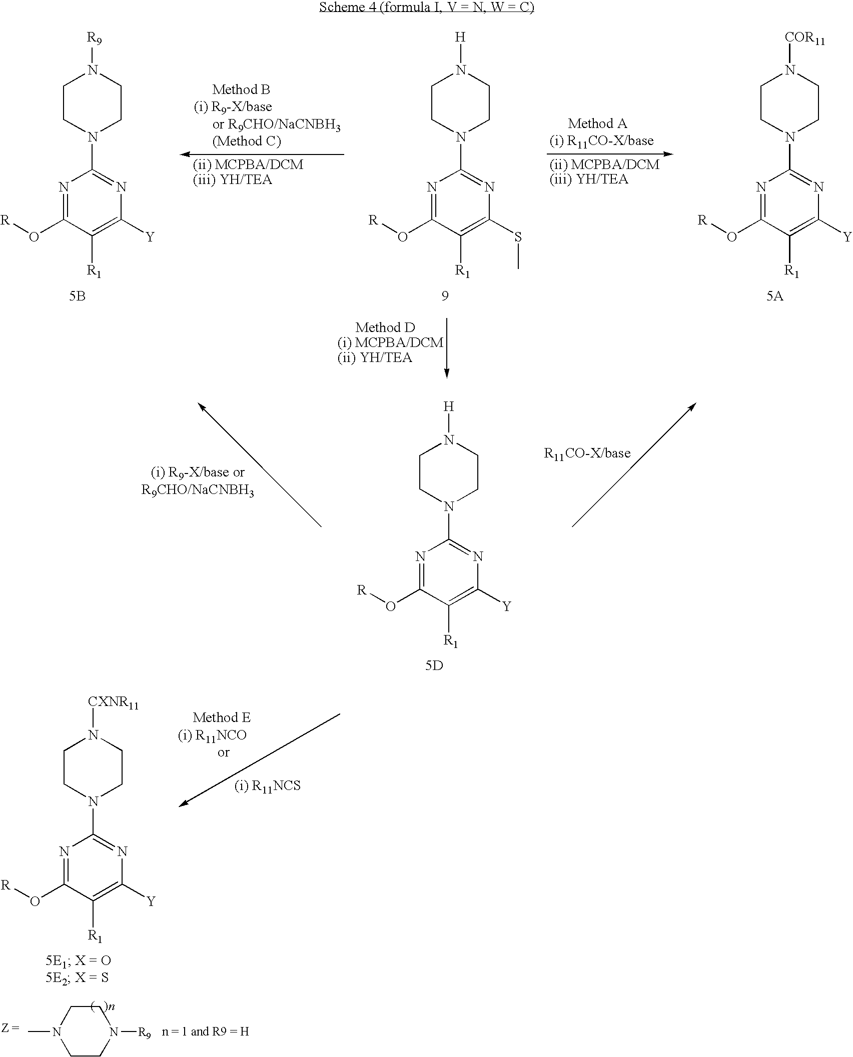

Secondary amine derivatives of 5 may be prepared from the unsubstituted amine 5 as shown in Scheme 4. For example, compound 9 can be acylated with the requisite acid chloride (R₁₁COX) to give the acyl derivative of the amine intermediate. The amine intermediate can be treated with MCPBA to form the reactive species that can be subsequently treated with the requisite amine (YH) to give the desired product 5A (Method A). In another example compound 9 can be allowed to react with alkyl halide (R₉X) in presence of base to form the alkyl intermediate (Method B). The alkyl intermediate can be also obtained from 9 by reductive amination through use of requisite aldehyde (R₉CHO, Method C)). The reductive amination step can be carried out through use of a suitable hydride reagent under appropriate conditions, e.g., NaBH₄ at room temperature under an inert atmosphere. Alternatively, cyanoborohydride or triacetoxyborhydride can be used under appropriate conditions. The amine intermediate can be treated with MCPBA to form the reactive species that can be subsequently treated with the requisite amine (YH) to give the desired product 5 B (Method A). Alternatively in Method D compound 9 can be first converted to the reactive species by the treatment with MCPBA followed by treatment with the amine (YH) to give compound 5 D. Compound 5 D can be subsequently subjected to acylation (R₁₁COX), alkylation (R₉X) or reductive amination (R₉CHO) to give the target compounds 5 A and 5, respectively. Compound 5 can be also allowed to react with the requisite isocyanate or isothiocyanate to give the target compounds 5 E1 and 5 E2, respectively (Method E).

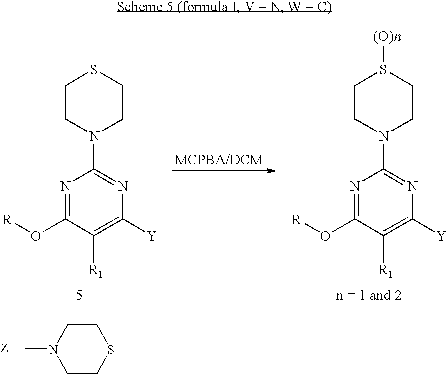

Compound 5 where Z is thiomorpholine can be converted to the corresponding thioxo and thione products by treatment with MCPBA in CH₂Cl₂ at room temperature according to Scheme 5.

Some compounds of this invention may have one or more asymmetric centers and are typically depicted in the form of racemic mixtures. This invention encompass racemic mixtures, partially racemic mixtures and separate enantiomers and diasteromers.

“Pharmaceutically-acceptable salts” include anions of inorganic and organic acids, including hydrochloric acid, hydrobromic acid, sulfuric acid, phosphoric acid, methanesulfonic acid, ethanesulfonic acid, malic acid, acetic acid, oxalic acid, tartaric acid, citric acid, lactic acid, fumaric acid, succinic acid, maleic acid, salicylic acid, benzoic acid, phenylacetic acid, mandelic acid, and such cations as alkaline, alkaline earth, ammonium, quaternary ammonium cations.

Pharmaceutical compositions including the inventive compounds can be pre-pared by well-known methods, such as those discussed in US 20060058308, which is incorporated by reference.

### EXAMPLES

The inventive compounds may be synthesized through use of standard procedures and techniques known in the art.

In addition, the acylation of amines to form amides through use of coupling agents is well known and is a convenient method used in peptide synthesis. The reaction entails mixing a coupling reagent with a suitable acid to form an anhydride that reacts with the amine to form the amide. Particularly suitable coupling reagents include N,N-dicyclohexylcarbodiimide and 1-hydroxybenzotriazole, use of either of which minimizes nitrile and lactam formation. Other coupling agents are well-known and can be used.

### General Procedures

All procedures were carried out at room temperature unless otherwise stated. N,N-Dimethylformamide (DMF) was dried over 4 \acute{Å} molecular sieves. Other commercially available reagents and solvents were used without further purification unless otherwise stated. Organic solvent extracts were dried over anhydrous MgSO₄. ¹H NMR spectra wre recorded on Bruker WM300 instrument through use of CDCl₃, DMSO, MeOD or D₂O, unless otherwise stated. LC-MS were recorded on Agilent 1100 through use of CH₃CN/H₂O gradient with 0.1% TFA. For TLC analysis, Merck precoated TLC plates (silica gel 60 F 254, d=0.25 mm) were used. Flash chromatography was performed on silica through use of Teledyne Isco CombiFlash system.

### Example 1

### Formula I, V═C, W═N

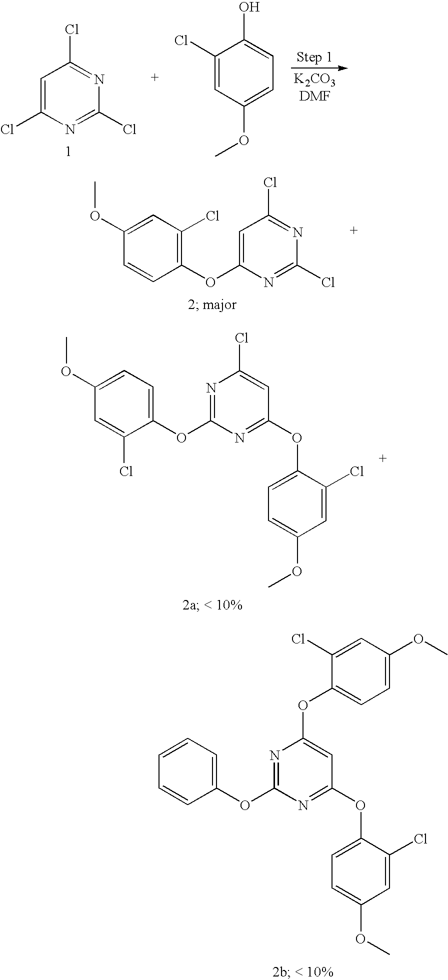

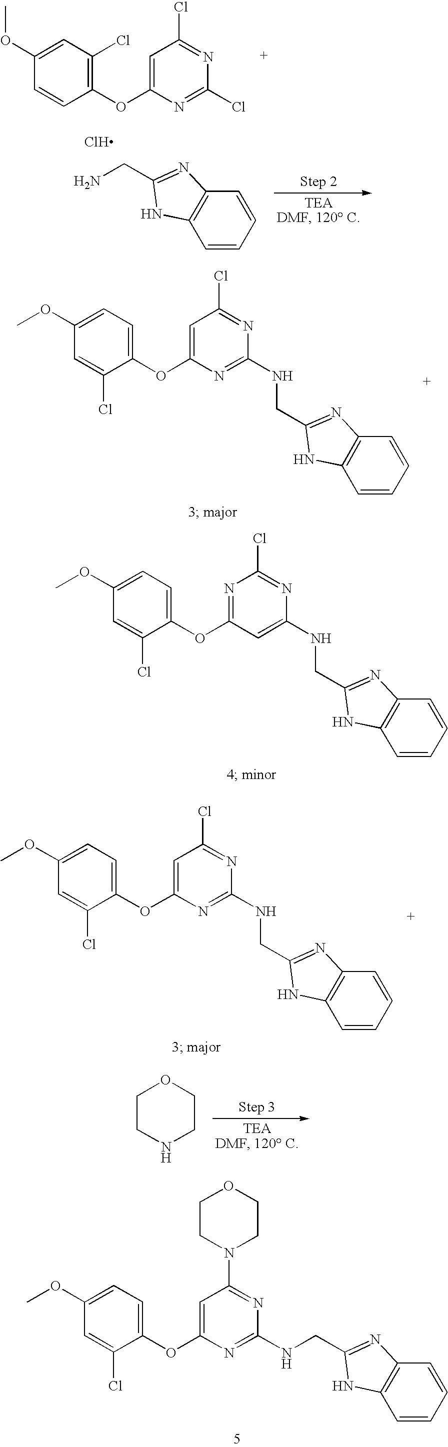

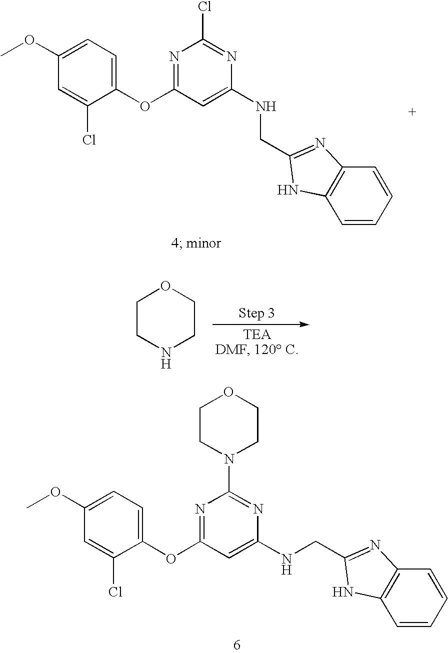

### Example 1

### Step 1. 2,6-Dichloro-4-(2-chloro-4-methoxyphenoxy)pyrimidine

To a solution of 2,4,6-trichloropyrimidine (3.8 g, 21.3 mmoles) in 150 mL of acetone at 0° C., was slowly added a solution of 2-chloro-4-methoxyphenoxide that was prepared by dissolving 2-chloro-4-methoxyphenol (3.7 g, 23.3 mmoles) and sodium hydroxide (1.0 g, 25.0 mmoles) in water (40 mL). A white precipitate formed rapidly and the mixture was slowly warmed to room temperature and stirred for an additional 3 hours. After dilution with water (150 mL) the crude product was extracted with CH₂ Cl₂ (3×100 mL). The combined organic extracts were dried over MgSO₄, filtered, and concentrated under reduced pressure. The crude product was purified by flash chromatography give 5.1 g (80%) as a white solid. LCMS: 306 (M+H)⁺.

### Step 2. 2-Chloro-4-(2-chloro-4-methoxyphenoxy)-6-[(1H-benzoimidazol-2-ylmethyl)-amino]pyrimidine and 6-chloro-4-(2-chloro-4-methoxyphenoxy)-2-[(1H-benzoimidazol-2-ylmethyl)-amino]pyrimidine

2,6-dichloro-4-(2-chloro-4-methoxyphenoxy)pyrimidine (1.5 g, 4.9 mmoles) was dissolved in DMF (50 mL) and 2-(aminomethyl)benzimidazole dihydrochloride hydrate (1.2 g, 5.5 mmoles), triethylamine (0.8 mL, 8.2 mmoles) was added. The mixture was heated at 120° C. for 2 hours with stirred under N₂. After completion of the reaction, an equal volume of water was added with cooling. the crude product was extracted with EtOAc (3×50 mL). The combined organic extracts were dried over MgSO₄, filtered, and concentrated under reduced pressure. The crude product was purified by flash chromatography give 1.56 g (78%) of 2-chloro-4-(2-chloro-4-methoxyphenoxy)-6-[(1H-benzoimidazol-2-ylmethyl)-amino]pyrimidine and the other isomer 6-Chloro-4-(2-chloro-4-methoxyphenoxy)-2-[(1H-benzoimidazol-2-ylmethyl)-amino]pyrimidine 300 mg (15%). LCMS: 417 (M+H)⁺.

### Step 3. 2-[4-[(1H-Benzimidazol-2-ylmethyl)-amino]-6-(2-chloro-4-methoxyphenoxy)-pyrimidin-2-yl]-morpholine (100340)

To a solution of 6-chloro-4-(2-chloro-4-methoxyphenoxy)-2-[(1H-benzoimidazol-2-ylmethyl)-amino]pyrimidine (200 mg, 0.5 mmoles), morpholine (83 mg, 1.0 mmoles), DMF (4 mL) in a 10 mL microwave vial was added TEA (0.1 mL, 1.0 mmoles). The solution was degassed with N₂ for 10 min before being capped and heated in the microwave reactor for 10 min at 120° C. Once complete, the reaction was diluted with 1 N NaOH (10 mL) and EtOAc (50 mL). The EtOAc layer was separated, dried over MgSO₄, filtered, and concentrated under reduced pressure. The crude product was submitted for flash chromatography purification. the title compound was obtained in (85 mg, 85% yield). LCMS: 467 (M+H)⁺. (M⁺¹=467.10, Retention time=2.52; 5-99% CH₃CN/H₂O gradient with 0.01% TFA).

### 4-[4-[(1H-Benzimidazol-2-ylmethyl)-amino]-6-(2-chloro-4-methoxy-phenoxy)-pyrimidin-2-yl]-morpholine (100341)

Compound 6 was prepared from 6-chloro-4-(2-chloro-4-methoxyphenoxy)-2-[(1H-benzoimidazol-2-ylmethyl)-amino]pyrimidine (200 mg, 0.5 mmoles by a similar process to that described above, to afford the title compound (81 mg, 80% yield). LCMS: 467 (M+H)⁺. (M⁺¹=467.23, Retention time=2.55; 5-99% CH₃ CN/H₂O gradient with 0.01% TFA).

### Example 2 a

### Formula I, V═C, W═N

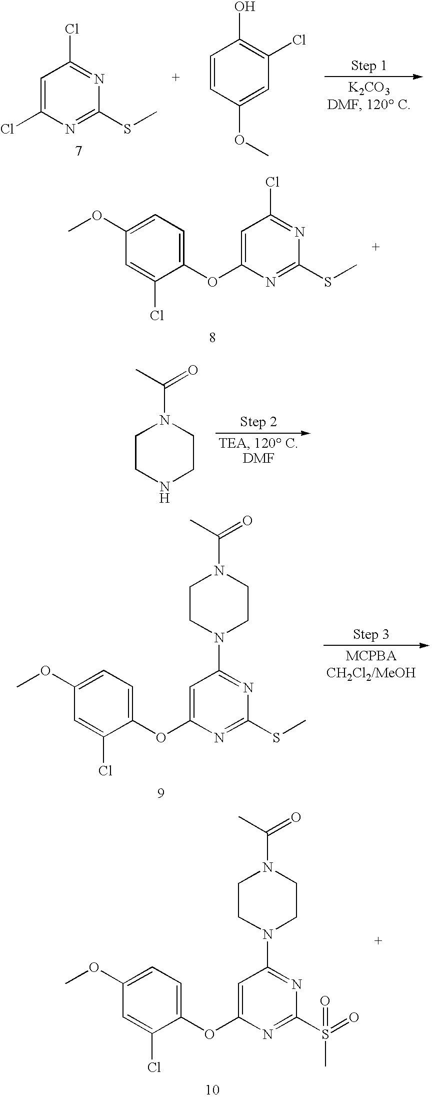

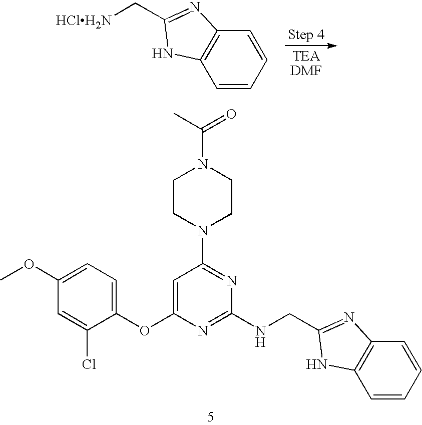

### Example 2 b

### Formula I, V═C, W═N

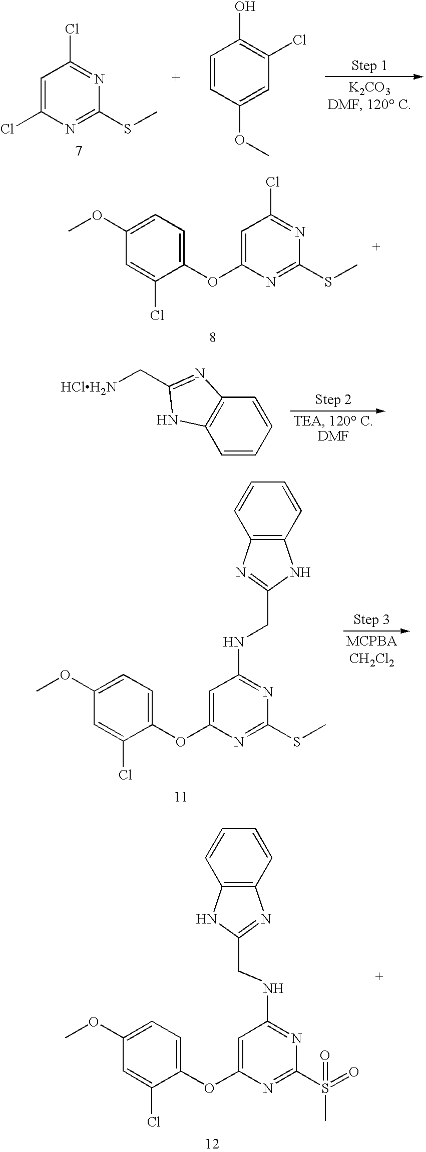

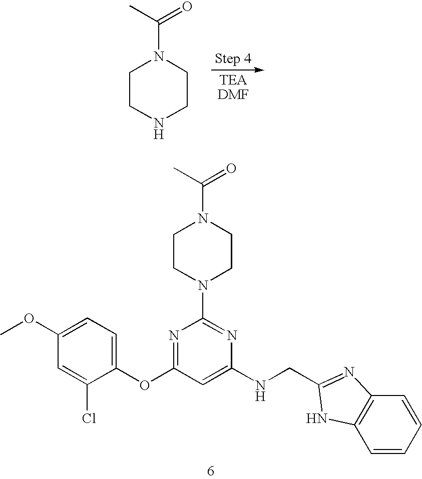

### Step 1. 6-Chloro-4-(2-chloro-4-methoxyphenoxy)-2-(methylthio)pyrimidine

4,6-dichloro-2-(methylthio)pyrimidine (5.3 g, 27 mmoles) was dissolved in DMF (50 mL) and 2-chloro-4-methoxyphenol (4.3 g, 27 mmoles), K₂CO₃ (5 g, 40 mmoles) was added. The mixture was stirred under N₂ at 80° C. for 1 h. After completion of the reaction, an equal volume of water was added with cooling. the crude product was extracted with CH₂Cl₂ (3×100 mL). The combined organic extracts were dried over MgSO₄ filtered, and concentrated under reduced pressure. The crude product was purified by flash chromatography to give 7.8 g (91%) of 6-chloro-4-(2-chloro-4-methoxyphenoxy)-2-(methylthio)pyrimidine. LCMS: 318 (M+H)⁺.

### Step 2. 6-[(1H-benzoimidazol-2-ylmethyl)-amino]-4-(2-chloro-4-methoxyphenoxy)-2-(methylthio)pyrimidine

6-chloro-4-(2-chloro-4-methoxyphenoxy)-2-(methylthio)pyrimidine (3.1 g, 9.8 mmoles) was dissolved in DMF (50 mL) and 2-(aminomethyl)benzimidazole dihydrochloride hydrate (2.5 g, 11.4 mmoles), triethylamine (1.7 mL, 16 mmoles) was added. The mixture was stirred under N₂ at 120° C. for 2 h. After completion of the reaction, an equal volume of water was added with cooling. the crude product was extracted with EtOAc (3×100 mL). The combined organic extracts were dried over MgSO₄, filtered, and concentrated under reduced pressure. The crude product was purified by flash chromatography give 3.7 g (90%) of 6-[(1H-benzoimidazol-2-ylmethyl)-amino]-4-(2-chloro-4-methoxyphenoxy)-2-(methylthio)pyrimidine. LCMS: 428 (M+H)⁺.

### Step 3. 6-[(1H-benzoimidazol-2-ylmethyl)-amino]-4-(2-chloro-4-methoxyphenoxy)-2-(methylsulfonyl)pyrimidine

6-[(1H-benzoimidazol-2-ylmethyl)-amino]-4-(2-chloro-4-methoxyphenoxy)-2-(methylthio)pyrimidine (1.9 g, 4.6 mmoles) was dissolved in CH₂Cl₂ (50 mL) and MeOH (50 mL), m-chloroperbenzoic acid (77%) (3 g, 16 mmoles) was added. The mixture was stirred at room temperature for 30 mins and concentrated. Aqueous sodium hydroxide (1 M, 100 mL) was added and the crude product was extracted with CH₂Cl₂ (3×50 mL). The combined organic extracts were dried over MgSO₄, filtered, and concentrated under reduced pressure. The crude product was purified by flash chromatography give 1.83 g (90%) of a brown solid as compound 12, 6-[(1H-benzoimidazol-2-ylmethyl)-amino]-4-(2-chloro-4-methoxyphenoxy)-2-(methylsulfonyl)pyrimidine. LCMS: 460 (M+H)⁺.

### Step 4. 1-{4-[4-[(1H-benzimidazol-2-ylmethyl)amino]-6-(2-chloro-4-methoxyphenoxy)pyrimidin-2-yl]-piperazin-1-yl}ethanone

To a solution of 6-[(1H-benzoimidazol-2-ylmethyl)-amino]-4-(2-chloro-4-methoxyphenoxy)-2-(methylsulfonyl)pyrimidine (300 mg, 0.65 mmoles), 1-acetyl piperazine (125 mg, 0.98 mmoles), DMF (4 mL) in a 10 mL microwave vial was added TEA (0.66 mL, 0.65 mmoles). The solution was degassed with N₂ for 10 min before being capped and heated in a microwave reactor for 10 min at 120° C. Once complete, the reaction was diluted with 1 M NaOH (10 mL) and EtOAc (20 mL). The EtOAc layer was separated, dried over MgSO₄, filtered, and concentrated under reduced pressure. The crude product was submitted for flash chromatography purification. the title compound was obtained in (298 mg, 90% yield). LCMS: 508 (M+H)⁺. (M⁺¹=508.09, Retention time=2.52; 5-99% CH₃ CN/H₂ 0 gradient with 0.01% TFA).

The compounds listed in Table 1 and Table 2 were prepared in similar fashion.

TABLE 1
(formula I, V = C—R₁, W = N) Analogs of compound 5 (X = O, R¹ = H)

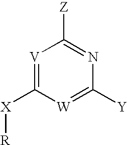

| | | | | ESMS |
|---|---|---|---|---|
| Compound # | R | Z | Y | M + H |
| 5.01 | 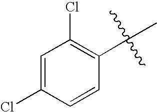 | 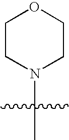 | 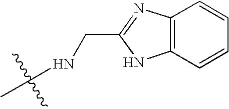 | 472.43 |
| 5.02 | 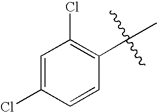 | 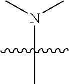 | 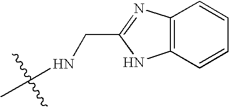 | 430.23 |
| 5.03 | 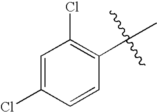 | 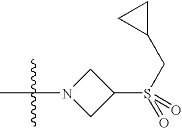 | 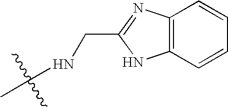 | 560.10 |
| 5.04 | 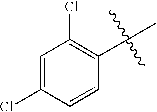 | 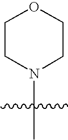 | 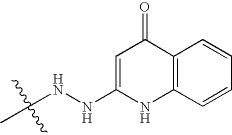 | 500.09 |
| 5.05 | 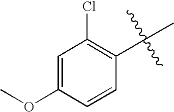 | 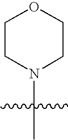 | 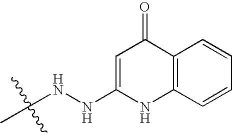 | 495.15 |
| 5.06 | 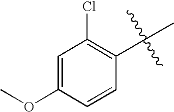 | 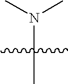 | 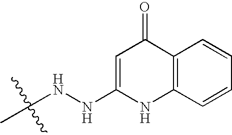 | 453.14 |
| 5.07 | 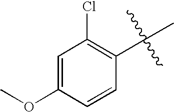 | 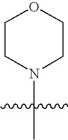 | 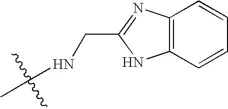 | 467.10 |
| 5.08 | 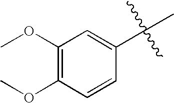 | 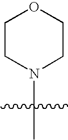 | 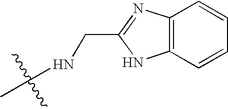 | 463.14 |
| 5.09 | 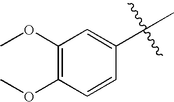 | 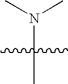 | 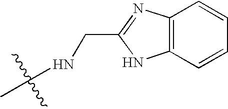 | 421.31 |
| 5.10 | 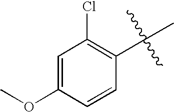 | 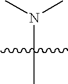 | 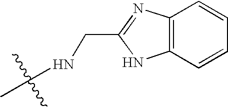 | 425.20 |
| 5.11 | 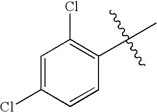 | 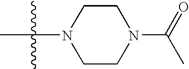 | 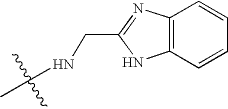 | 513.14 |
| 5.12 | 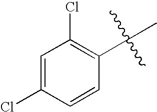 | 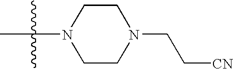 | 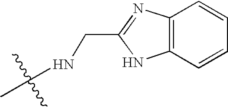 | 524.12 |
| 5.13 | 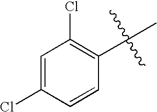 | 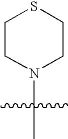 | 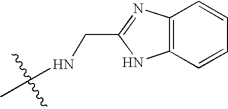 | 472.43 |
| 5.14 | 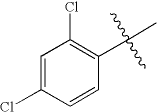 | 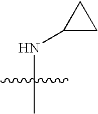 | 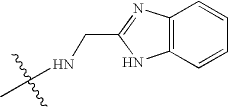 | 442.55 |
| 5.15 | 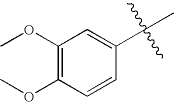 | 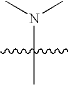 | 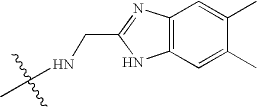 | 449.22 |
| 5.16 | 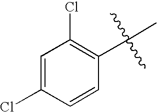 | 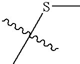 | 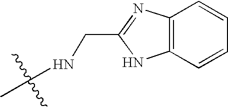 | 433.08 |
| 5.17 | 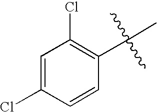 | 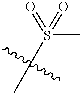 | 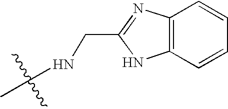 | 465.05 |
| 5.18 | 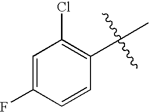 | 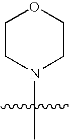 | 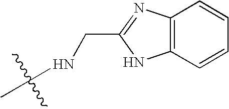 | 455.15 |
| 5.19 | 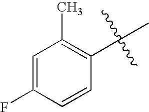 | 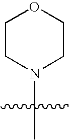 | 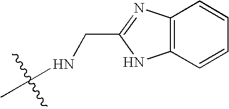 | 435.45 |
| 5.20 | 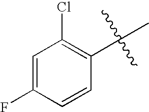 | 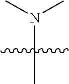 | 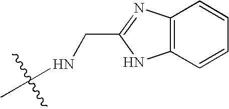 | 412.55 |
| 5.21 | 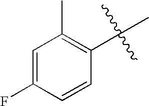 | 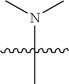 | 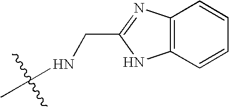 | 393.32 |
| 5.22 | 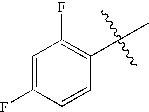 | 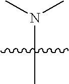 | 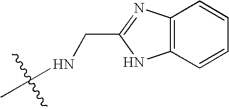 | 397.14 |
| 5.23 | 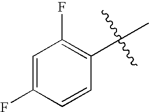 | 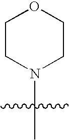 | 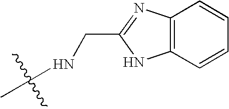 | 439.21 |
| 5.24 | 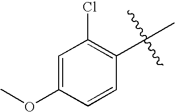 | 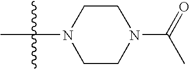 | 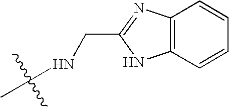 | 508.10 |
| 5.25 | 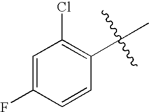 | 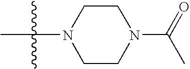 | 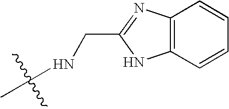 | 496.15 |
| 5.26 | 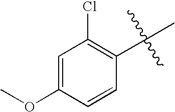 | 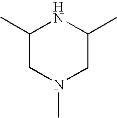 | 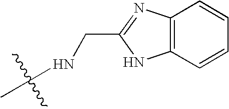 | 494.51 |
| 5.27 | 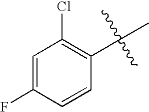 | 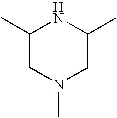 | 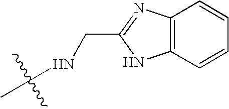 | 482.15 |
| 5.28 | 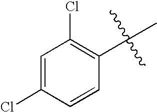 | 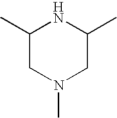 | 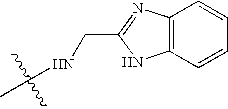 | 499.05 |
| 5.29 | 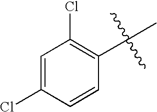 | 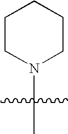 | 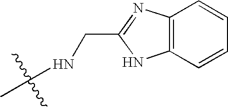 | 470.24 |
| 5.30 | 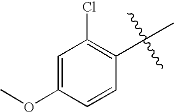 | 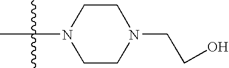 | 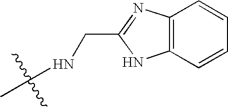 | 510.20 |
| 5.31 | 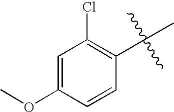 | 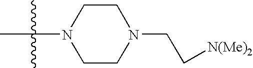 | 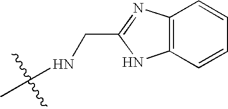 | 537.43 |
| 5.32 | 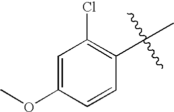 | 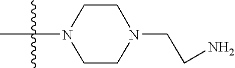 | 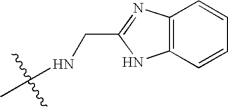 | 510.23 |
| 5.33 | 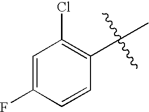 | 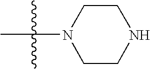 | 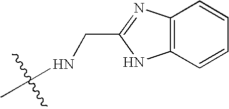 | 454.15 |
| 5.34 | 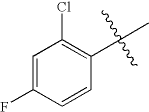 | 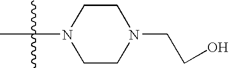 | 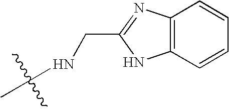 | 498.16 |
| 5.35 | 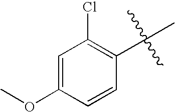 | 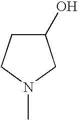 | 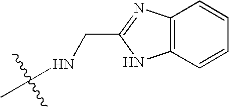 | 467.05 |
| 5.36 | 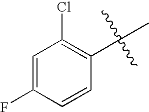 | 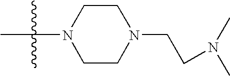 | 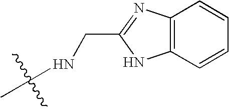 | 526.23 |
| 5.37 | 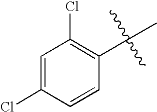 | 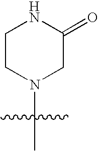 | 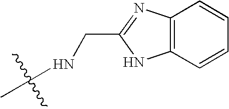 | 485.44 |
| 5.38 | 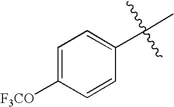 | 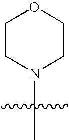 | 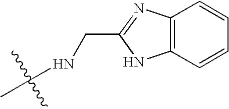 | 487.17 |
| 5.39 | 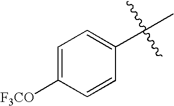 | 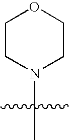 | 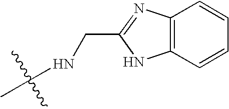 | 415.13 |
| 5.40 | 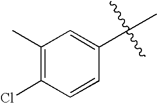 | 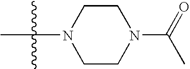 | 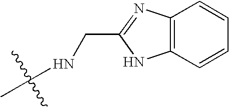 | 492.19 |
| 5.41 | 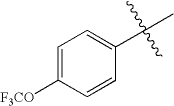 | 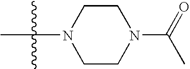 | 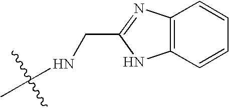 | 528.23 |
| 5.42 | 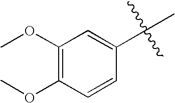 | 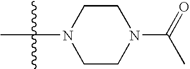 | 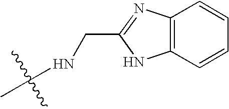 | 504.22 |
| 5.43 | 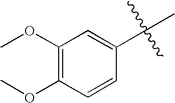 | 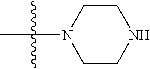 | 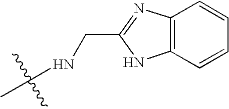 | 462.24 |
| 5.44 | 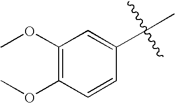 | 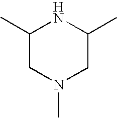 | 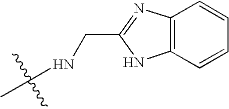 | 490.25 |
| 5.45 | 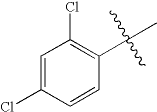 | 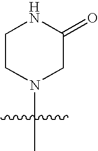 | 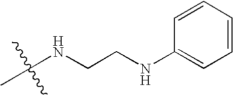 | 474.11 |
| 5.46 | 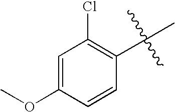 | 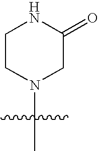 | 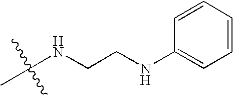 | 469.18 |
| 5.47 | 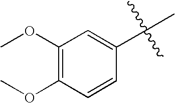 | 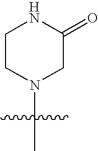 | 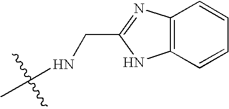 | 478.21 |
| 5.48 | 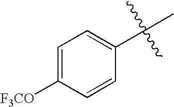 | 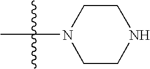 | 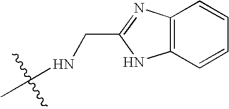 | 486.18 |
| 5.49 | 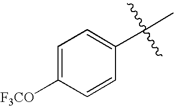 | 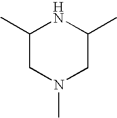 | 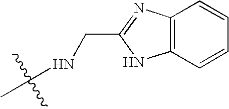 | 514.23 |
| 5.50 | 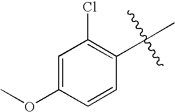 | 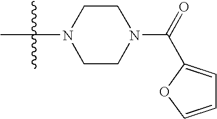 | 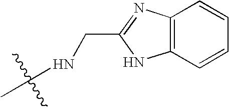 | 561.18 |
| 5.51 | 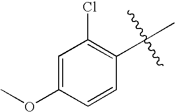 | 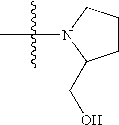 | 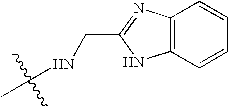 | 481.18 |
| 5.52 | 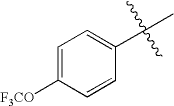 | 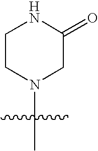 | 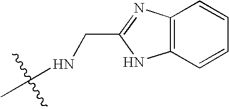 | 500.15 |
| 5.53 | 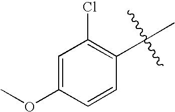 | 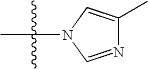 | 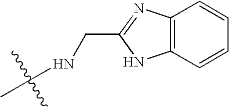 | 462.13 |
| 5.54 | 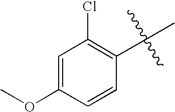 | 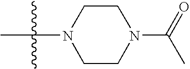 | 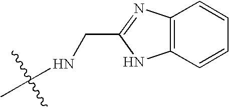 | 508.18 |
| 5.55 | 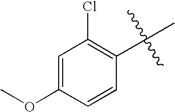 | 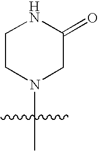 | 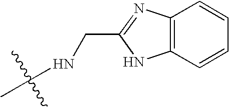 | 480.12 |
| 5.56 | 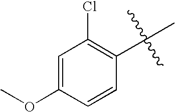 | 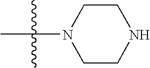 | 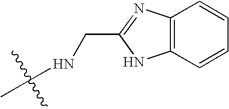 | 466.16 |
| 5.57 | 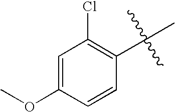 | 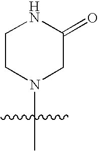 | 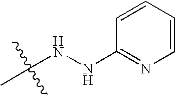 | 442.11 |
| 5.58 | 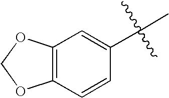 | 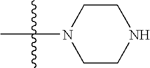 | 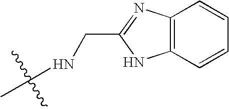 | 445.18 |
| 5.59 | 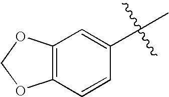 | 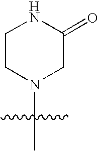 | 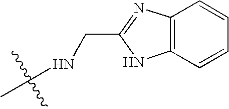 | 460.20 |
| 5.60 | 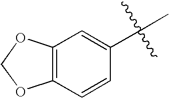 | 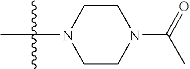 | 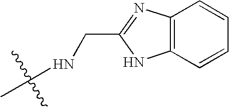 | 488.20 |
| 5.61 | 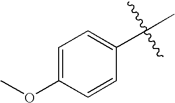 | 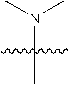 | 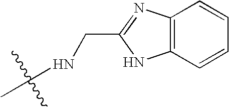 | 391.15 |
| 5.62 | 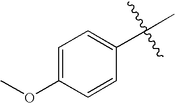 | 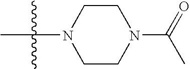 | 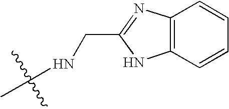 | 473.23 |
| 5.63 | 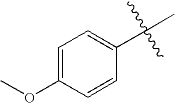 | 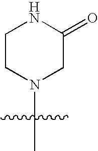 | 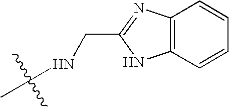 | 446.19 |
| 5.64 | 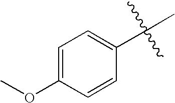 | 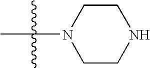 | 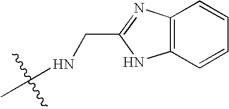 | 432.21 |
| 5.65 | 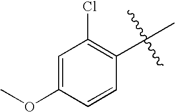 | 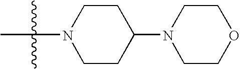 | 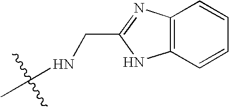 | 551.23 |
| 5.66 | 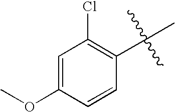 | 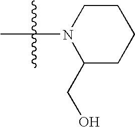 | 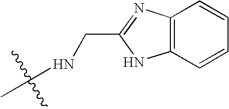 | 495.16 |
| 5.67 | 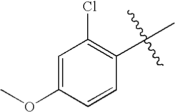 | 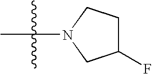 | 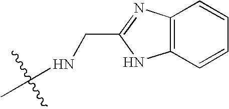 | 469.11 |
| 5.68 | 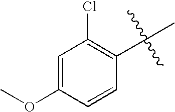 | 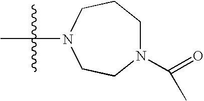 | 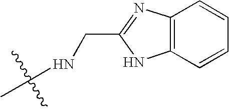 | 553.19 |
| 5.69 | 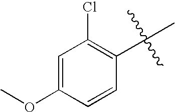 | 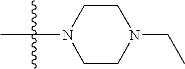 | 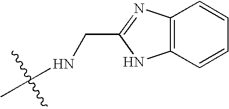 | 494.21 |
| 5.70 | 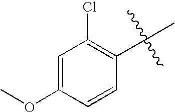 | 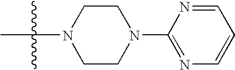 | 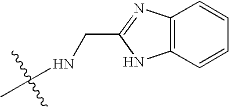 | 545.13 |
| 5.71 | 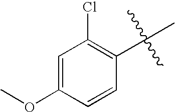 | 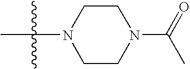 | 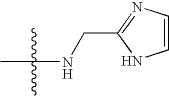 | 458.18 |
| 5.72 | 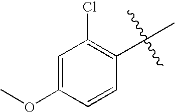 | 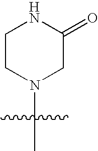 | 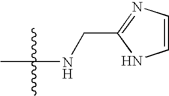 | 430.10 |
| 5.73 | 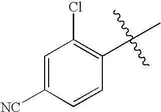 | 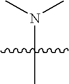 | 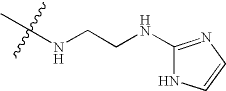 | 413.17 |

TABLE 2
(formula I, V = N, W = C—R₁) Analogs of compound 6 in Example 1 (X = O, R¹ = H)

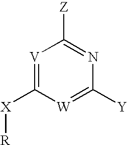

| | | | | ESMS |
|---|---|---|---|---|
| Compound # | R | Z | Y | M + H |
| 6.01 | 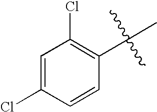 | 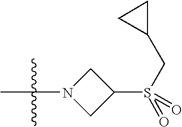 | 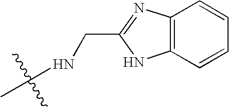 | 560.15 |
| 6.02 | 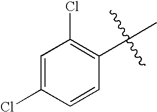 | 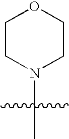 | 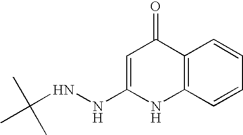 | 500.09 |
| 6.03 | 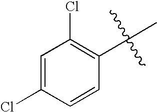 | 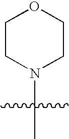 | 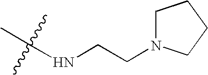 | 439.14 |
| 6.04 | 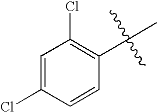 | 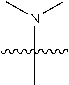 | 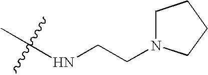 | 397.13 |
| 6.05 | 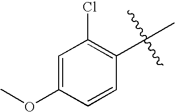 | 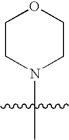 | 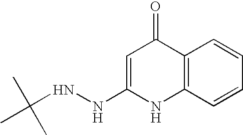 | 495.15 |
| 6.06 | 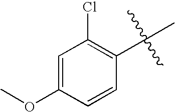 | 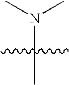 | 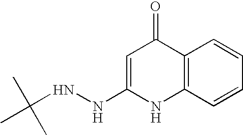 | 453.14 |
| 6.07 | 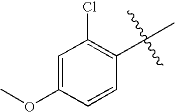 | 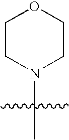 | 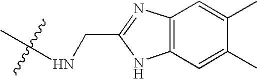 | 495.19 |
| 6.08 | 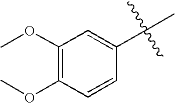 | 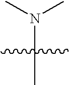 | 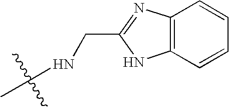 | 421.19 |
| 6.09 | 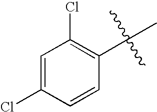 | 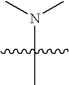 | 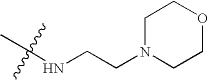 | 413.12 |
| 6.10 | 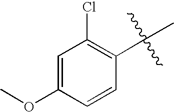 | 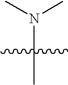 | 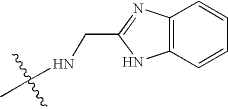 | 425.08 |
| 6.11 | 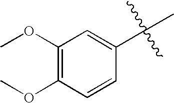 | 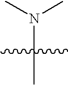 | 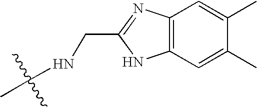 | 449.23 |
| 6.12 | 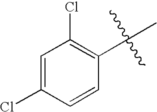 | H | 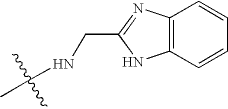 | 387.08 |
| 6.13 | 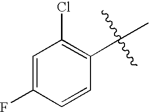 | 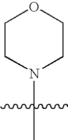 | 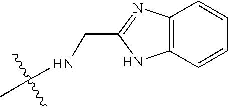 | 455.24 |
| 6.14 | 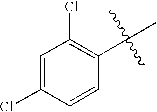 | 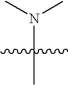 | 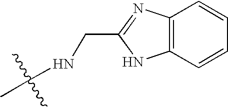 | 430.09 |
| 6.15 | 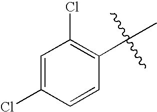 | 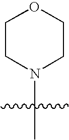 | 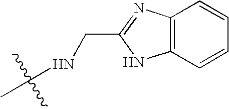 | 472.11 |
| 6.16 | 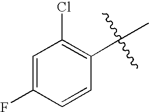 | 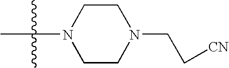 | 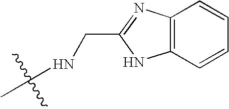 | |
| 6.17 | 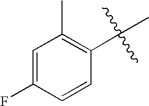 | 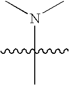 | 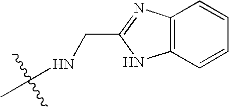 | 393.21 |
| 6.18 | 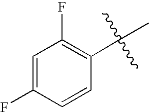 | 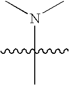 | 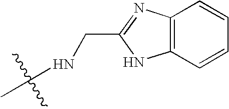 | 397.18 |
| 6.19 | 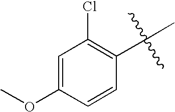 | 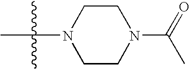 | 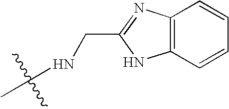 | 508.25 |
| 6.20 | 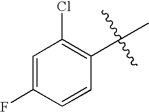 | 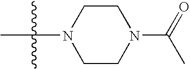 | 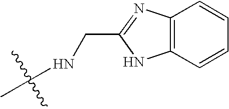 | 498.16 |
| 6.21 | 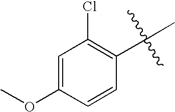 | 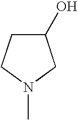 | 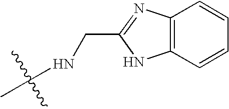 | 467.24 |
| 6.22 | 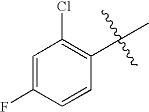 | 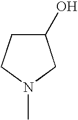 | 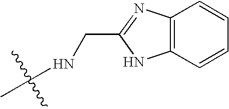 | 455.17 |
| 6.23 | 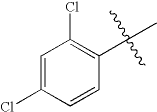 | 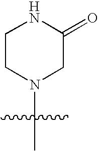 | 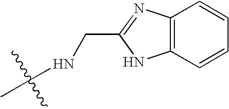 | 485.13 |
| 6.24 | 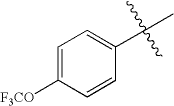 | 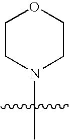 | 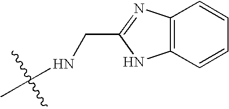 | 487.17 |
| 6.25 | 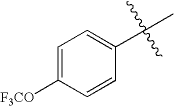 | 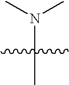 | 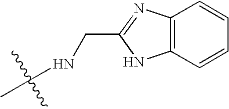 | 473.19 |
| 6.26 | 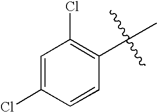 | H | 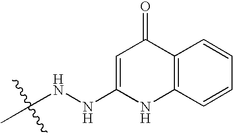 | 415.03 |
| 6.27 | 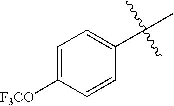 | 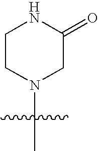 | 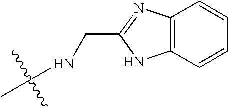 | 500.16 |
| 6.28 | 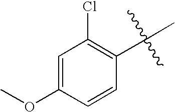 | H | 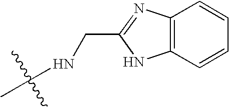 | 382.12 |
| 6.29 | 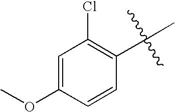 | 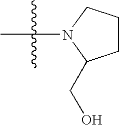 | 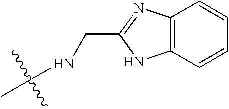 | 481.15 |
| 6.30 | 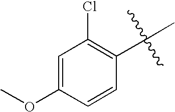 | 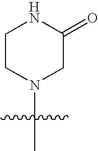 | 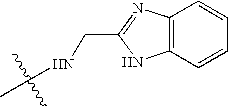 | 480.14 |
| 6.31 | 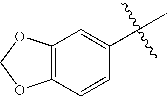 | 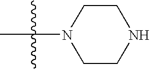 | 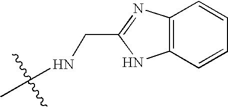 | 445.21 |
| 6.32 | 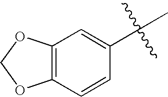 | 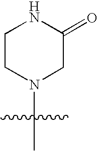 | 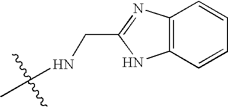 | 460.18 |
| 6.33 | 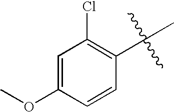 | NH₂ | 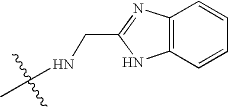 | |
| 6.34 | 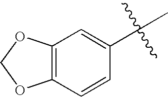 | 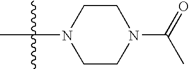 | 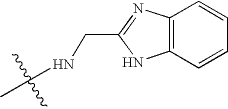 | 488.23 |
| 6.35 | 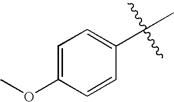 | 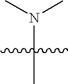 | 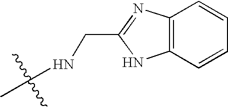 | 391.10 |
| 6.36 | 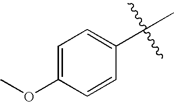 | 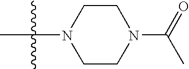 | 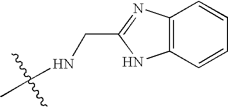 | 473.25 |
| 6.37 | 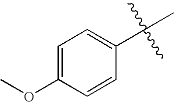 | 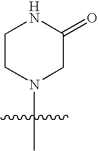 | 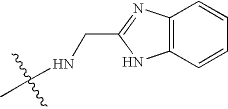 | 446.11 |
| 6.38 | 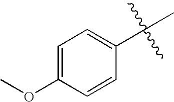 | 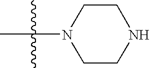 | 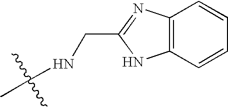 | 432.15 |
| 6.39 | 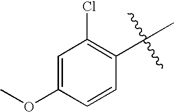 | 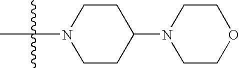 | 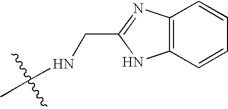 | 551.16 |
| 6.40 | 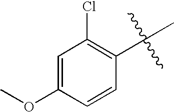 | 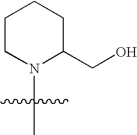 | 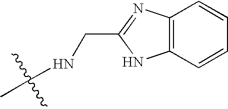 | 495.13 |
| 6.41 | 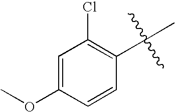 | 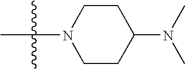 | 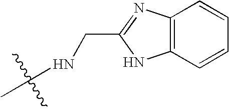 | 509.23 |
| 6.42 | 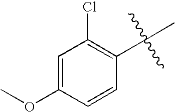 | 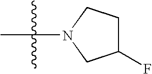 | 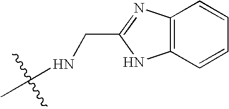 | 469.20 |
| 6.43 | 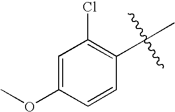 | 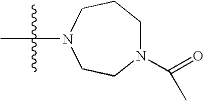 | 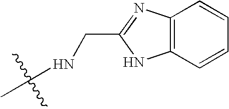 | 553.11 |
| 6.44 | 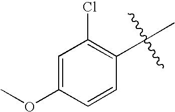 | 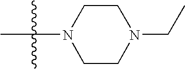 | 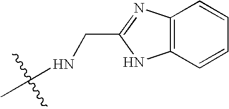 | 494.15 |
| 6.45 | 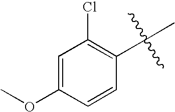 | 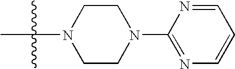 | 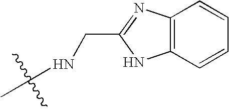 | 545.13 |
| 6.46 | 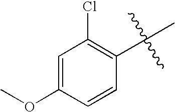 | 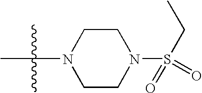 | 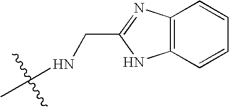 | 559.12 |
| 6.47 | 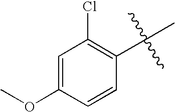 | 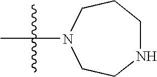 | 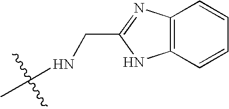 | 480.18 |
| 6.48 | 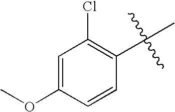 | 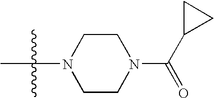 | 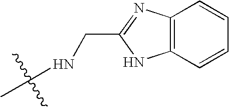 | 535.05 |
| 6.49 | 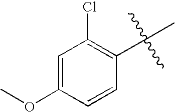 | 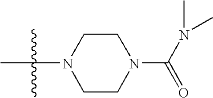 | 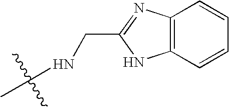 | 538.22 |
| 6.50 | 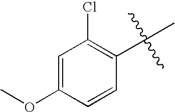 | 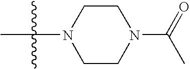 | 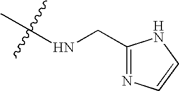 | 458.15 |
| 6.51 | 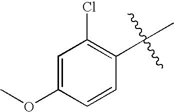 | 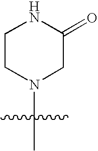 | 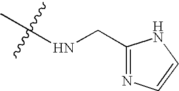 | 430.34 |
| 6.52 | 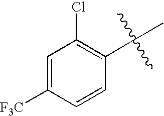 | 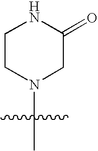 | 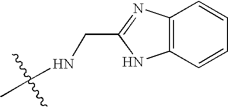 | 518.15 |
| 6.53 | 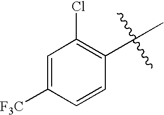 | 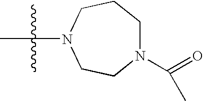 | 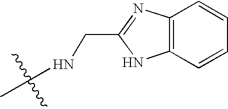 | 550.17 |
| 6.54 | 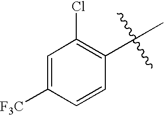 | 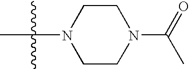 | 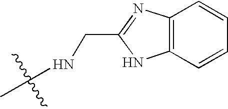 | 546.11 |
| 6.55 | 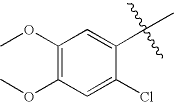 | 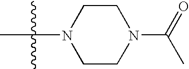 | 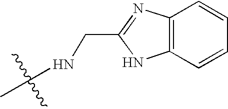 | 538.19 |
| 6.56 | 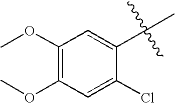 | 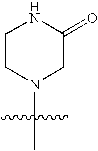 | 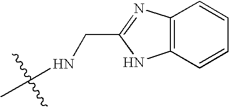 | 510.16 |
| 6.57 | 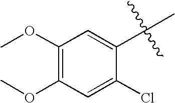 | 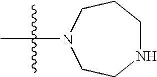 | 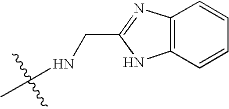 | 510.22 |
| 6.58 | 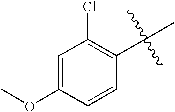 | 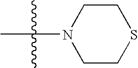 | 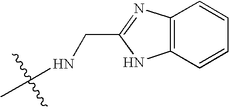 | 483.13 |
| 6.59 | 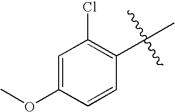 | 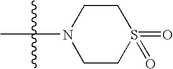 | 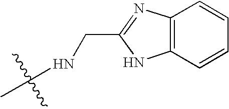 | 515.10 |
| 6.60 | 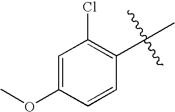 | 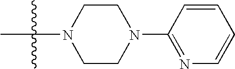 | 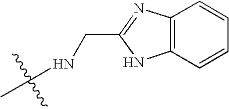 | 544.21 |
| 6.61 | 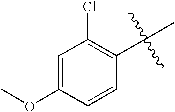 | 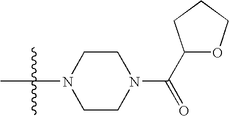 | 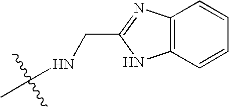 | 565.23 |
| 6.62 | 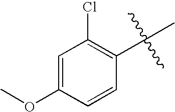 | 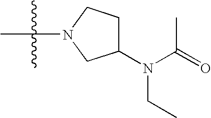 | 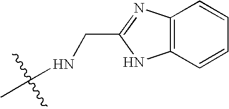 | 537.15 |
| 6.63 | 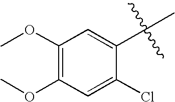 | 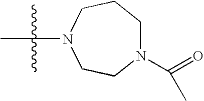 | 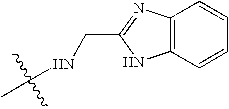 | 553.25 |
| 6.64 | 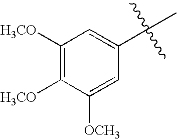 | 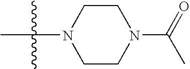 | 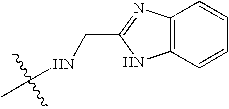 | 534.21 |
| 6.65 | 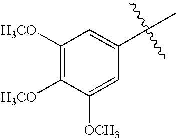 | 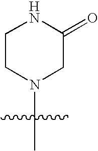 | 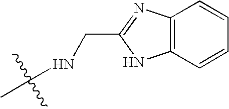 | 506.18 |
| 6.66 | 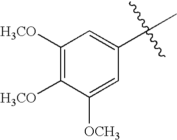 | 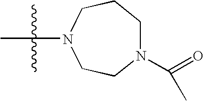 | 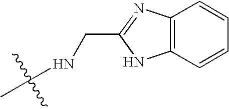 | 548.26 |
| 6.67 | 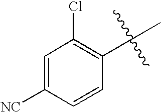 | 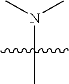 | 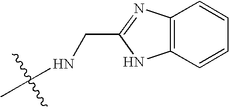 | 420.16 |
| 6.68 | 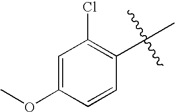 | 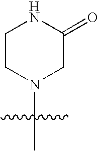 | 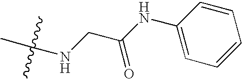 | |
| 6.69 | 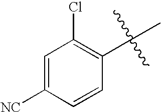 | 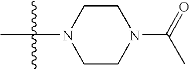 | 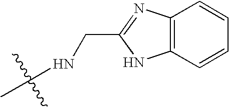 | 503.14 |
| 6.70 | 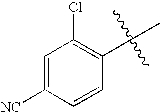 | 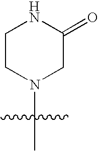 | 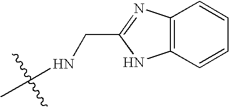 | 475.21 |
| 6.71 | 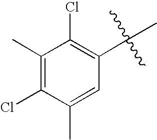 | 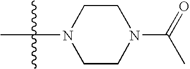 | 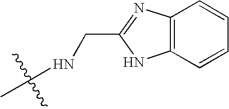 | 541.18 |
| 6.72 | 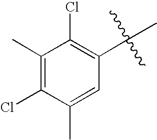 | 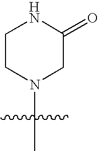 | 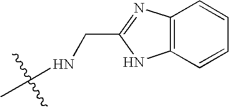 | 513.15 |
| 6.73 | 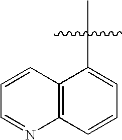 | 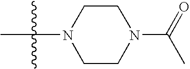 | 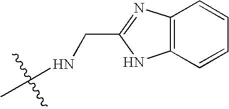 | 495.23 |
| 6.74 | 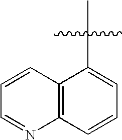 | 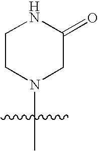 | 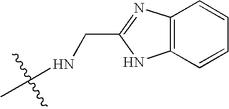 | 467.14 |
| 6.75 | 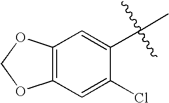 | 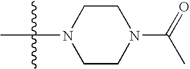 | 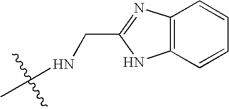 | 522.20 |
| 6.76 | 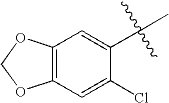 | 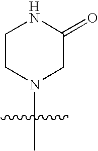 | 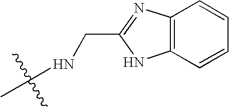 | 494.17 |
| 6.77 | 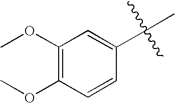 | 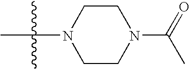 | 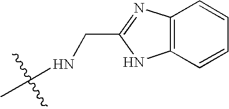 | 504.25 |
| 6.78 | 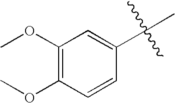 | 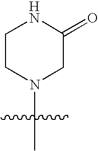 | 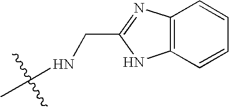 | 476.23 |
| 6.83 | 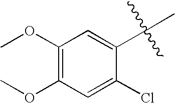 | 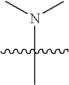 | 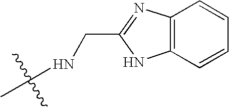 | 455.21 |
| 6.84 | 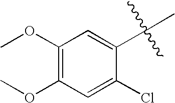 | 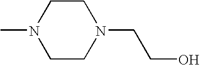 | 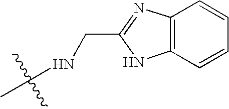 | 541.08 |
| 6.85 | 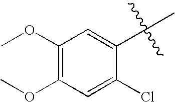 | 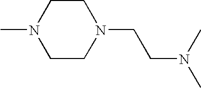 | 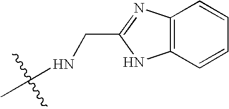 | 568.10 |
| 6.86 | 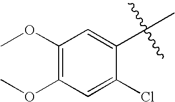 | 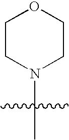 | 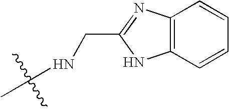 | 497.85 |
| 6.87 | 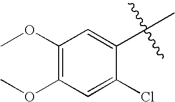 | 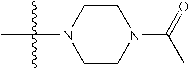 | 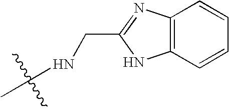 | 539.12 |
| 6.88 | 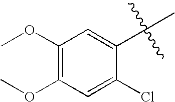 | 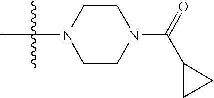 | 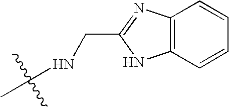 | 565.05 |
| 6.89 | 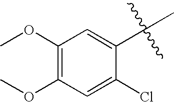 | 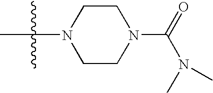 | 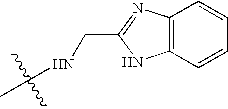 | 567.23 |
| 6.90 | 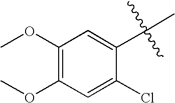 | 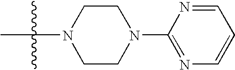 | 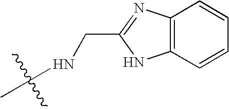 | 589.99 |
| 6.91 | 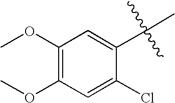 | 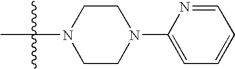 | 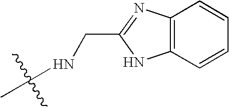 | 589.14 |
| 6.92 | 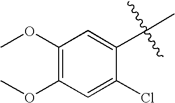 | 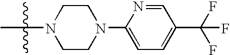 | 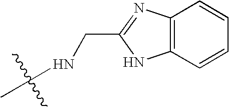 | 557.22 |
| 6.93 | 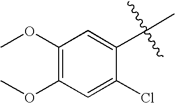 | 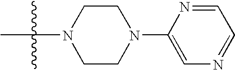 | 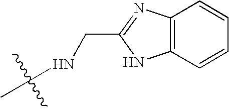 | 490.13 |
| 6.94 | 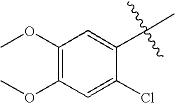 | 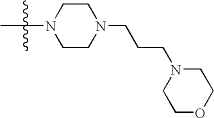 | 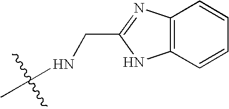 | 623.17 |
| 6.95 | 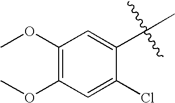 | 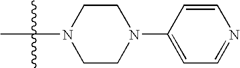 | 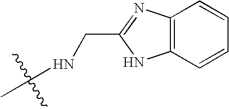 | 573.21 |
| 6.96 | 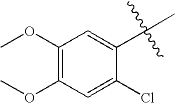 | 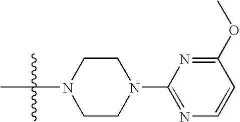 | 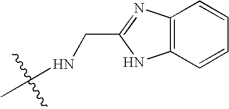 | 604.31 |
| 6.97 | 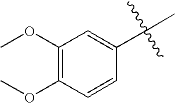 | 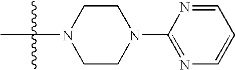 | 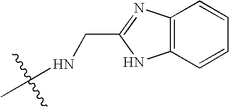 | 540.24 |
| 6.98 | 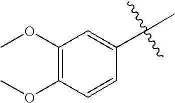 | 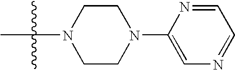 | 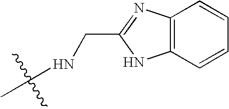 | 540.59 |
| 6.99 | 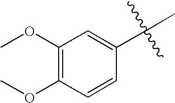 | 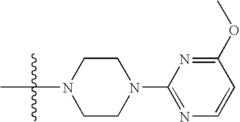 | 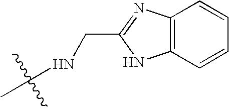 | 570.25 |
| 6.100 | 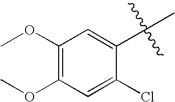 | 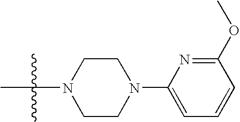 | 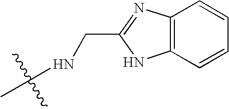 | 604.22 |
| 6.101 | 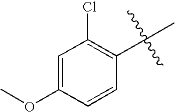 | 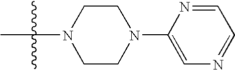 | 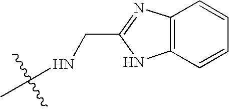 | 545.19 |
| 6.102 | 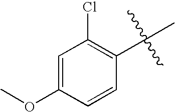 | 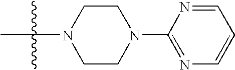 | 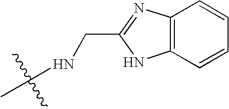 | 545.05 |
| 6.103 | 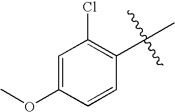 | 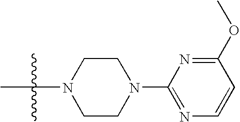 | 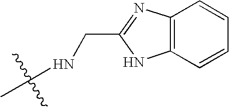 | 575.11 |
| 6.104 | 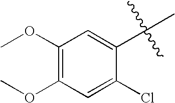 | 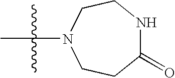 | 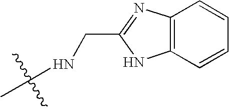 | 524.14 |
| 6.105 | 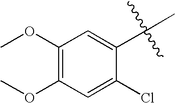 | 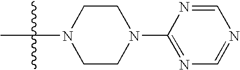 | 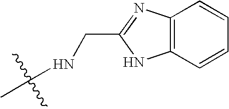 | 576.20 |
| 6.106 | 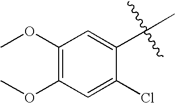 | 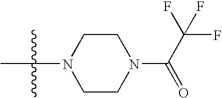 | 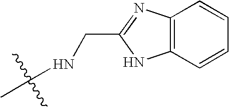 | 592.17 |
| 6.107 | 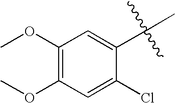 | 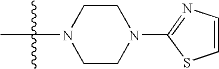 | 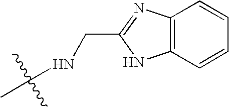 | 580.11 |
| 6.108 | 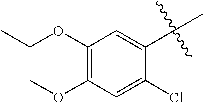 | 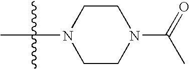 | 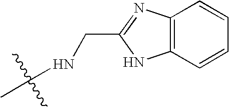 | 553.20 |
| 6.110 | 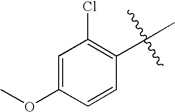 | 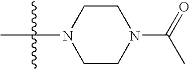 | 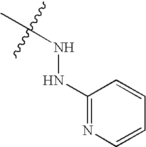 | 470.96 |
| 6.112 | 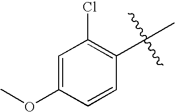 | 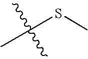 | 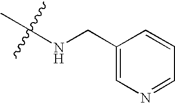 | 388.78 |
| 6.113 | 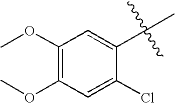 | 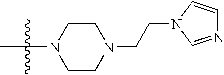 | 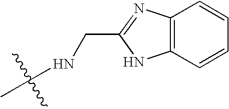 | 591.08 |
| 6.114 | 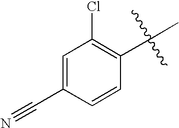 | 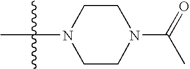 | 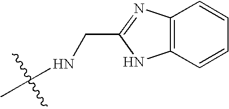 | 503.16 |
| 6.115 | 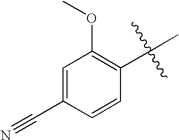 | 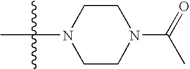 | 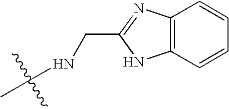 | 499.21 |
| 6.116 | 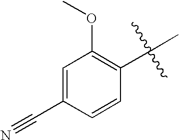 | 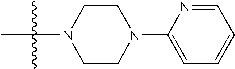 | 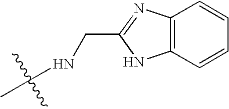 | 534.23 |
| 6.117 | 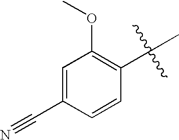 | 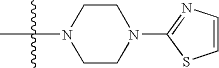 | 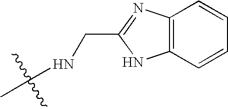 | 540.19 |
| 6.118 | 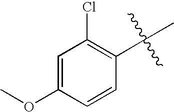 | 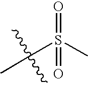 | 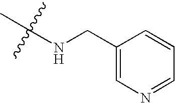 | 421.10 |
| 6.119 | 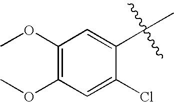 | 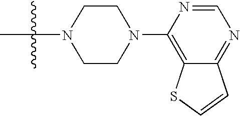 | 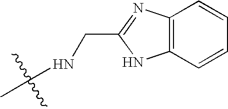 | 631.17 |
| 6.120 | 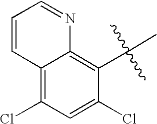 | 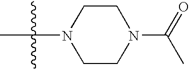 | 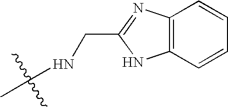 | 564.14 |
| 6.121 | 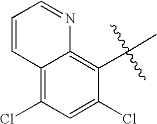 | 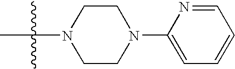 | 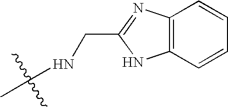 | 599.16 |
| 6.122 | 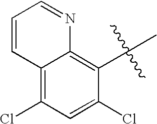 | 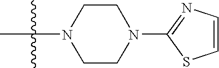 | 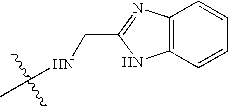 | 605.11 |
| 6.123 | 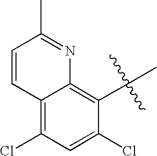 | 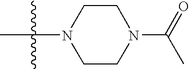 | 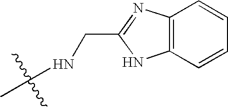 | 578.16 |
| 6.124 | 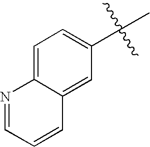 | 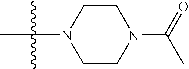 | 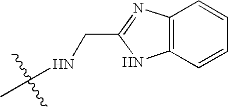 | 495.22 |
| 6.125 | 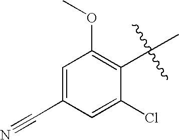 | 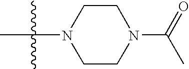 | 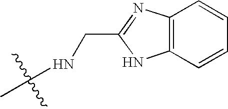 | 533.17 |
| 6.126 | 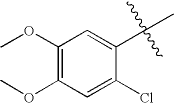 | 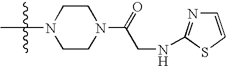 | 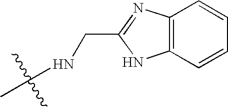 | 637.18 |
| 6.130 | 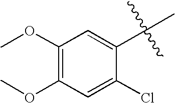 | 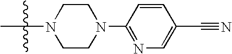 | 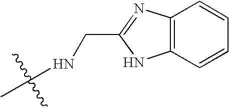 | 599.20 |
| 6.131 | 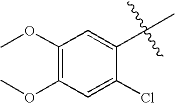 | 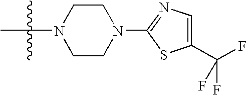 | 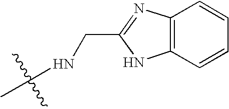 | 648.15 |
| 6.133 | 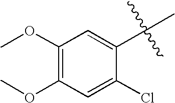 | 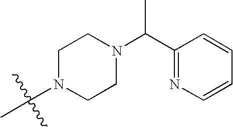 | 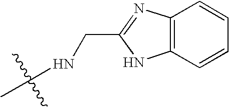 | 602.24 |
| 6.134 | 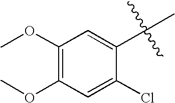 | 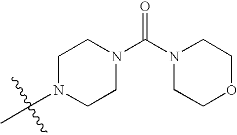 | 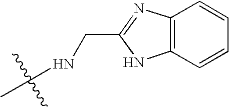 | 610.22 |
| 6.135 | 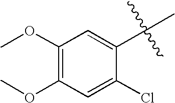 | 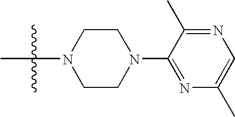 | 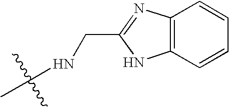 | 603.23 |
| 6.136 | 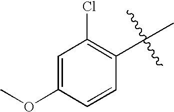 | 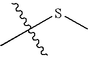 | 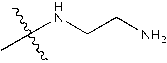 | 340.83 |
| 6.137 | 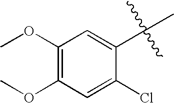 | 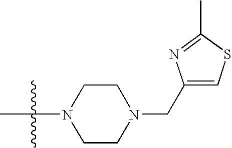 | 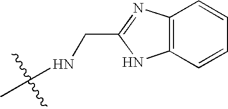 | 608.19 |
| 6.138 | 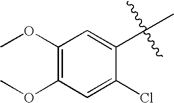 | 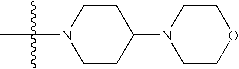 | 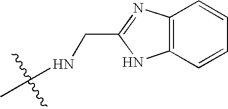 | 581.24 |
| 6.139 | 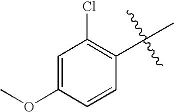 | 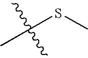 | 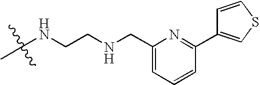 | 514.07 |
| 6.140 | 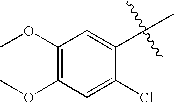 | 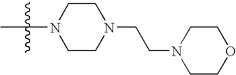 | 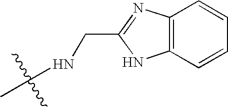 | 609.26 |
| 6.141 | 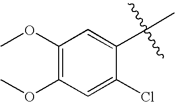 | 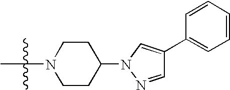 | 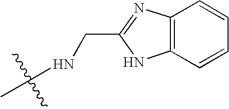 | 638.24 |
| 6.142 | 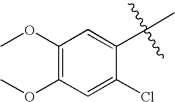 | 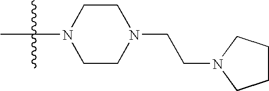 | 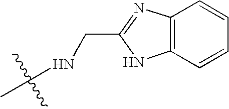 | 594.27 |
| 6.143 | 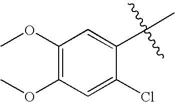 | 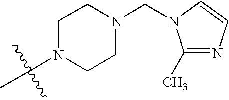 | 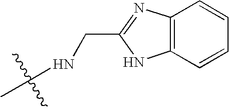 | 591.23 |
| 6.144 | 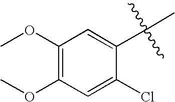 | 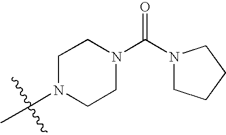 | 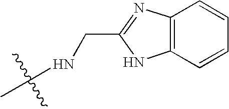 | 594.23 |
| 6.145 | 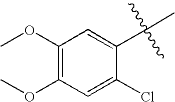 | 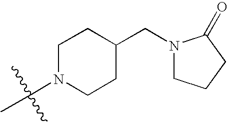 | 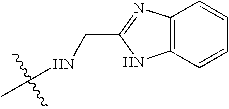 | 593.24 |
| 6.146 | 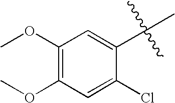 | 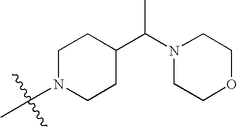 | 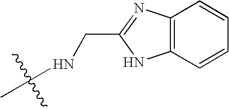 | 609.27 |
| 6.147 | 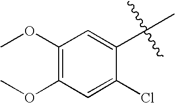 | 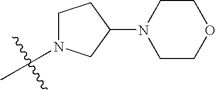 | 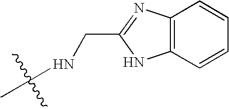 | 567.22 |
| 6.148 | 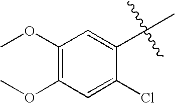 | 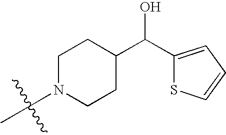 | 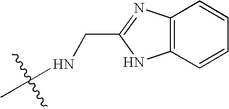 | 608.18 |
| 6.149 | 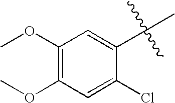 | 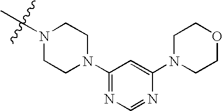 | 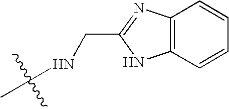 | 660.25 |
| 6.150 | 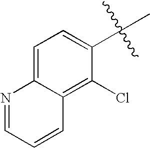 | 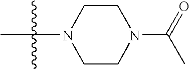 | 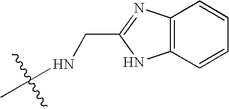 | 529.18 |

Preferred compounds include:

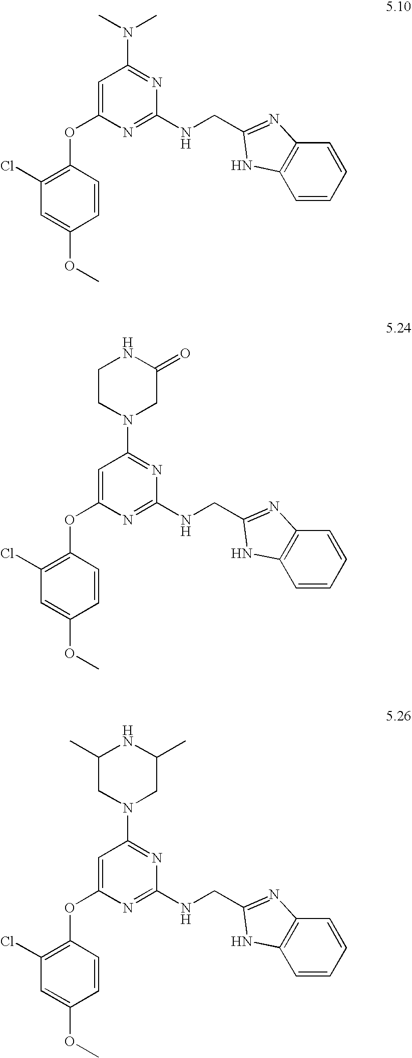

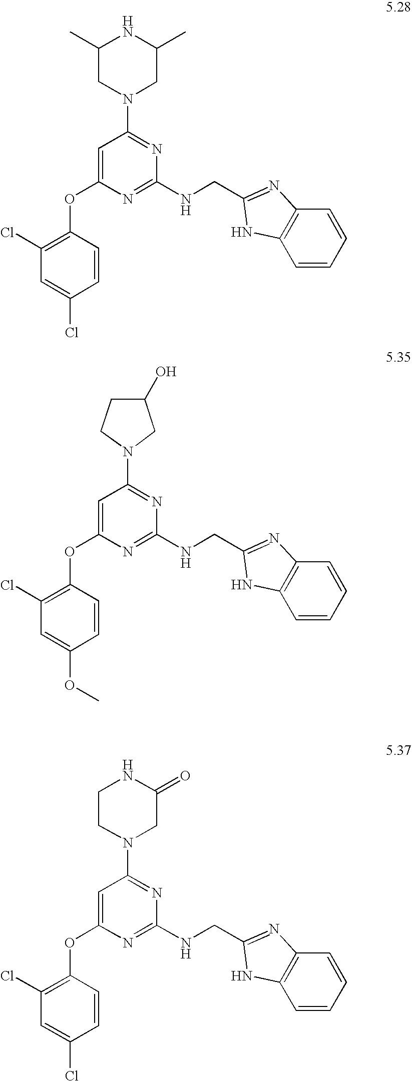

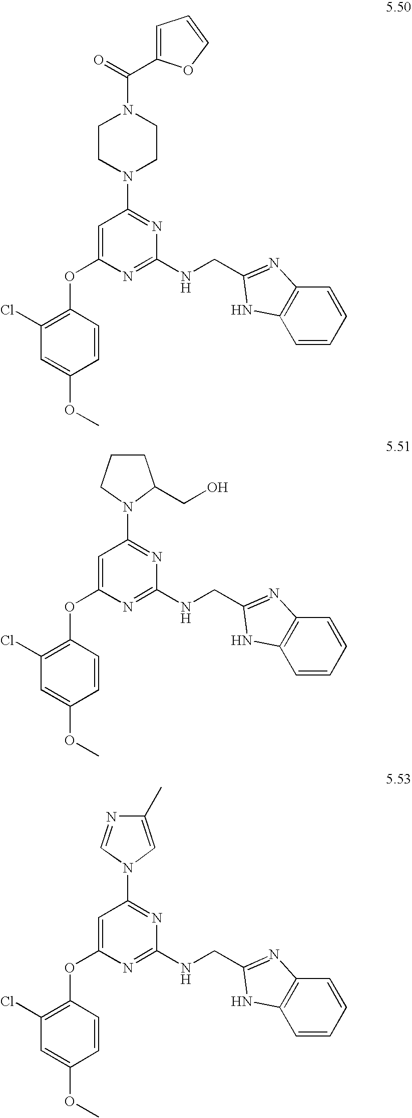

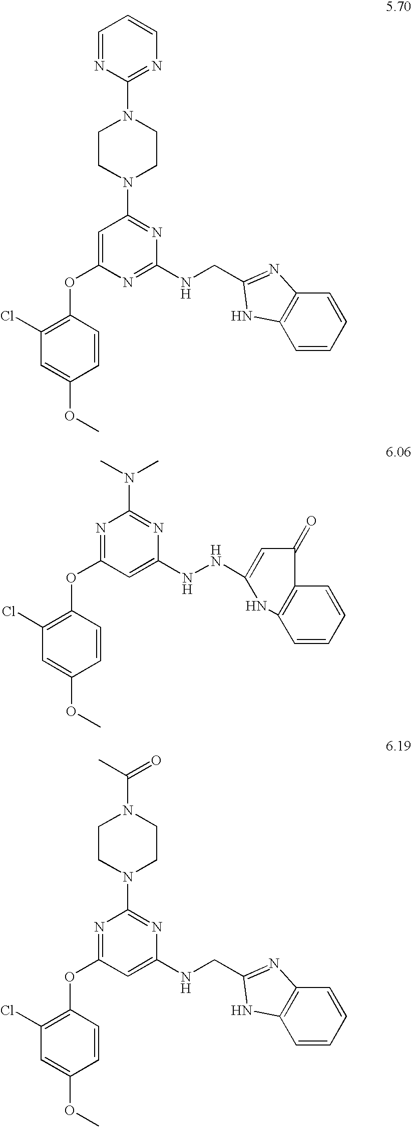

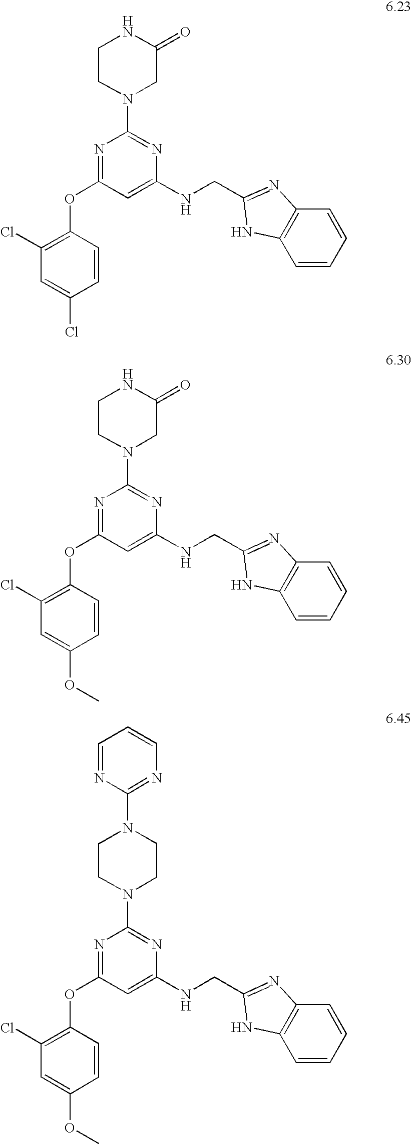

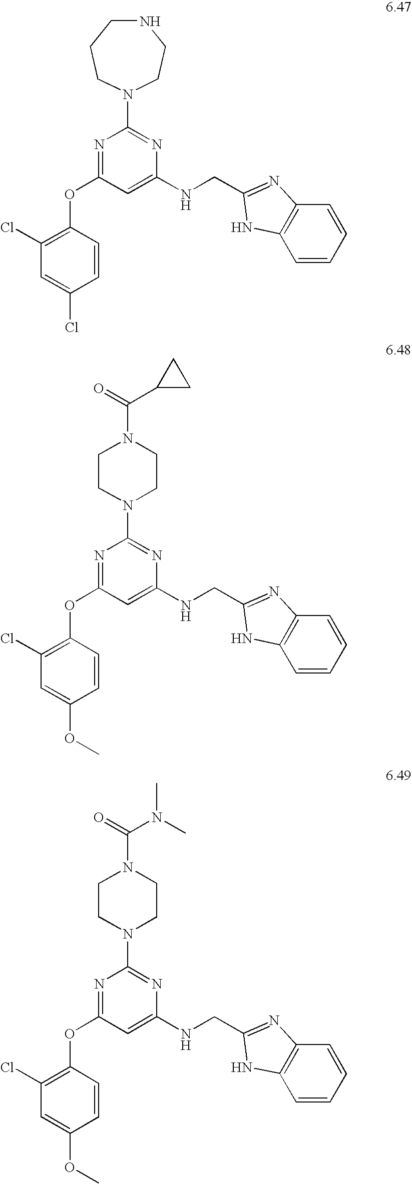

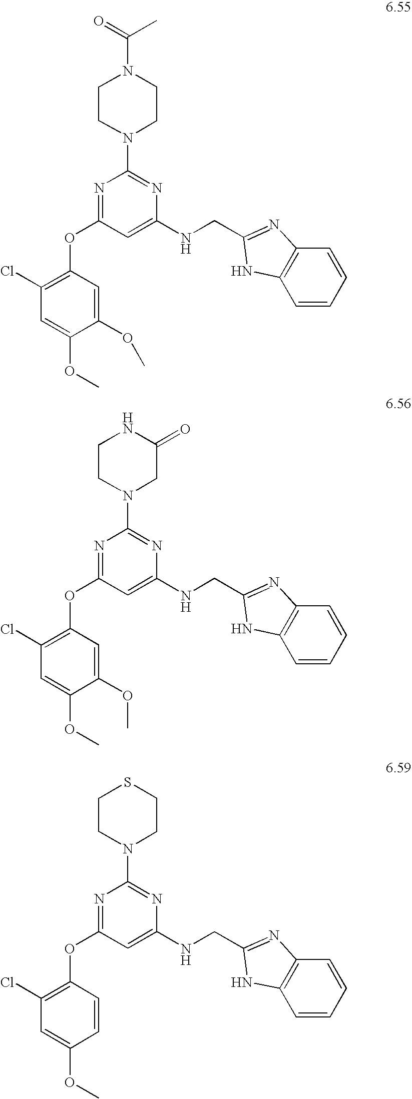

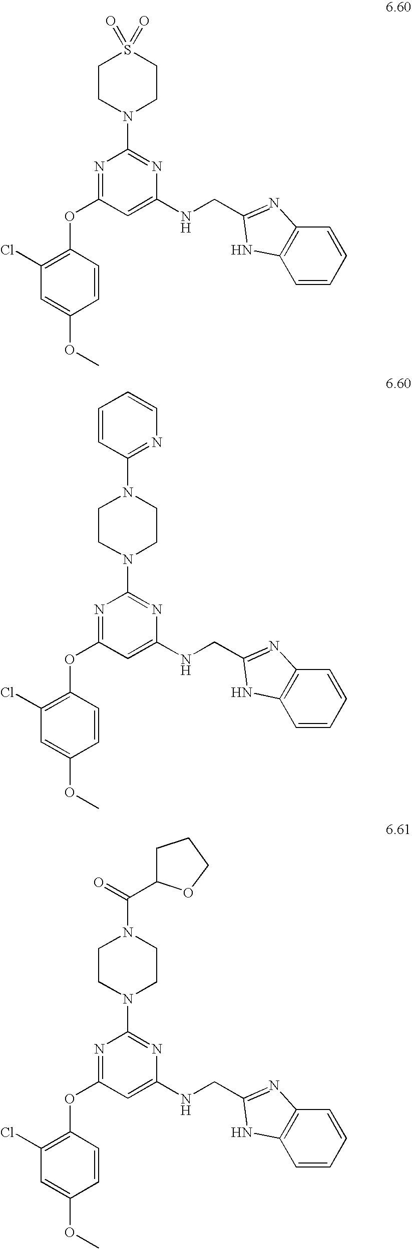

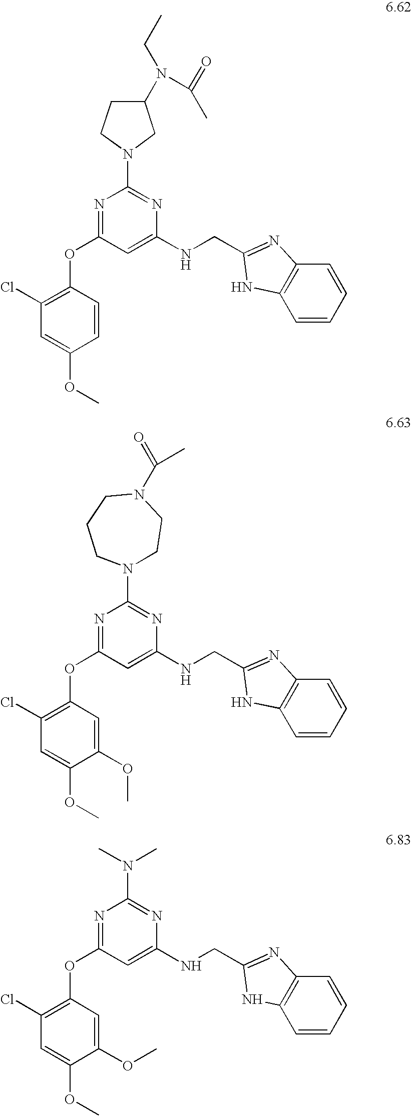

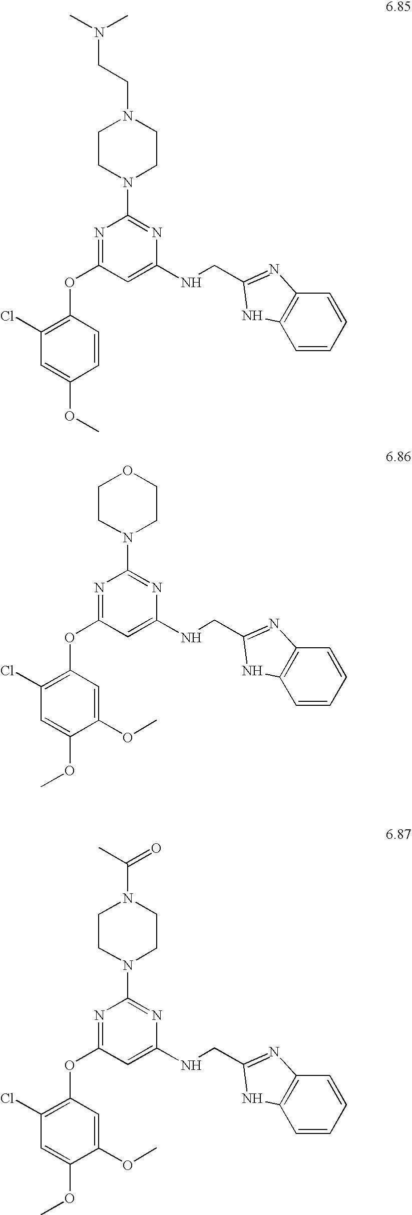

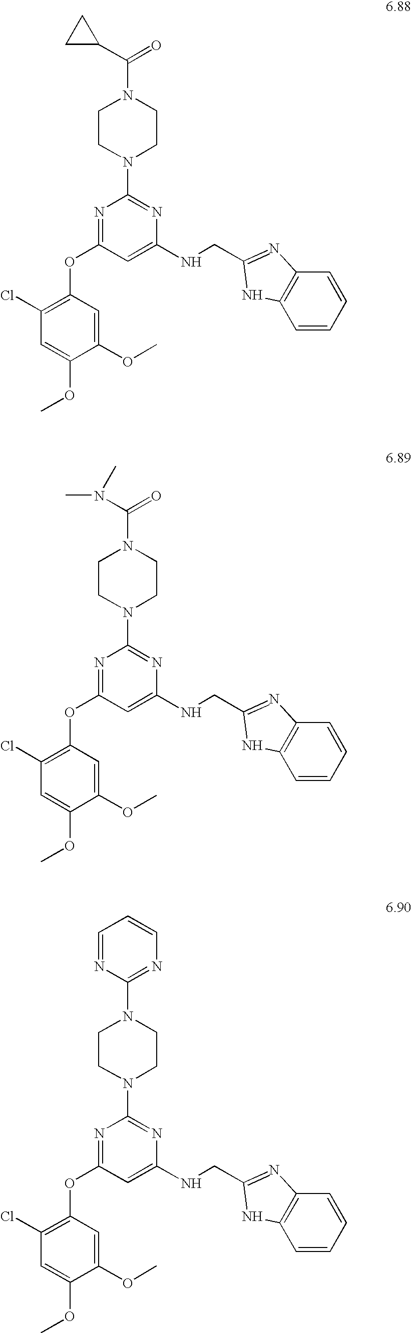

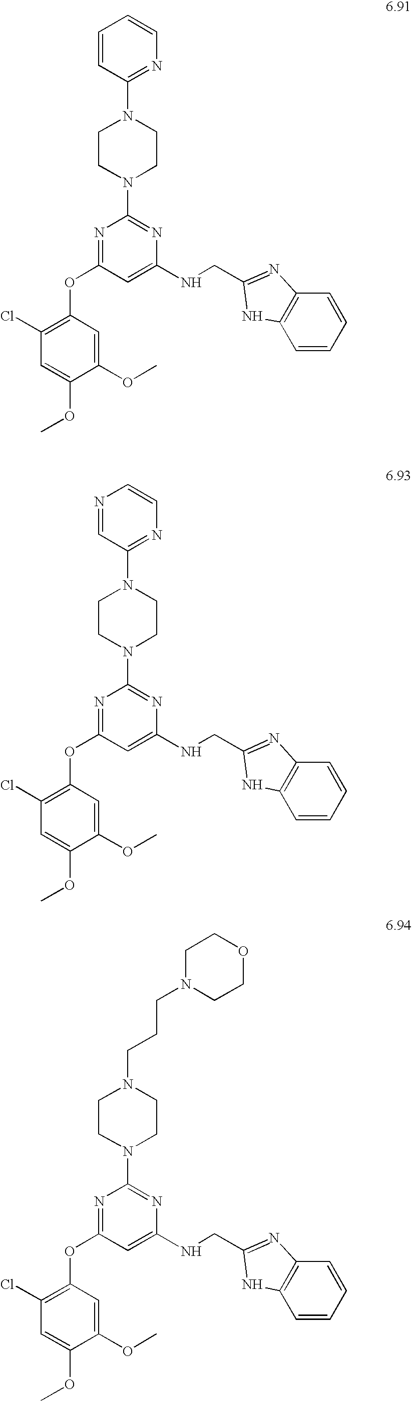

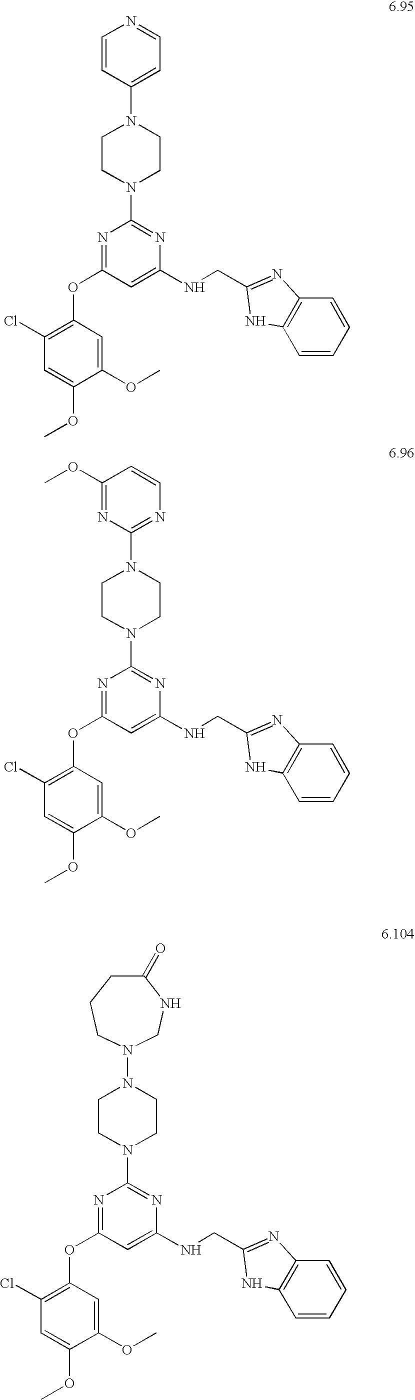

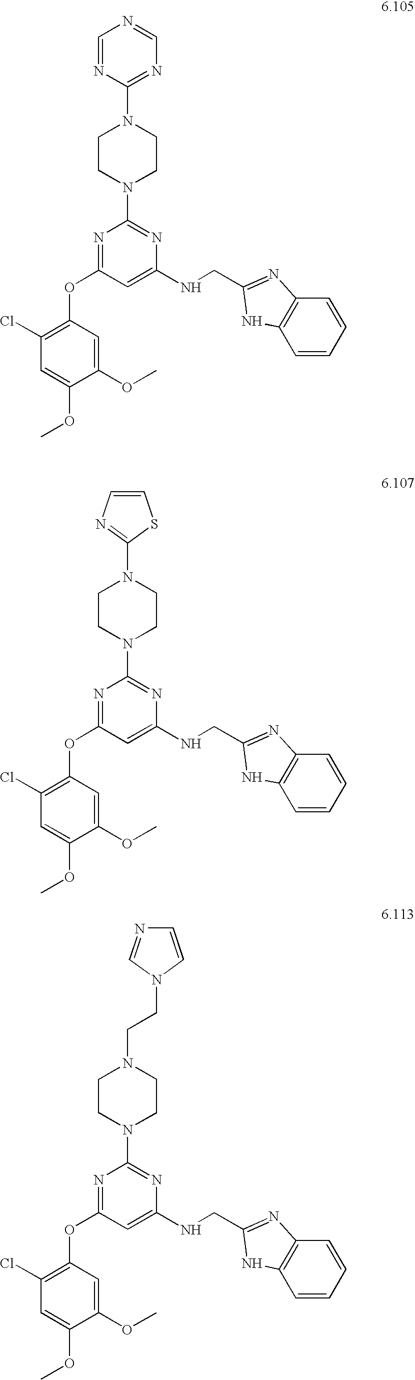

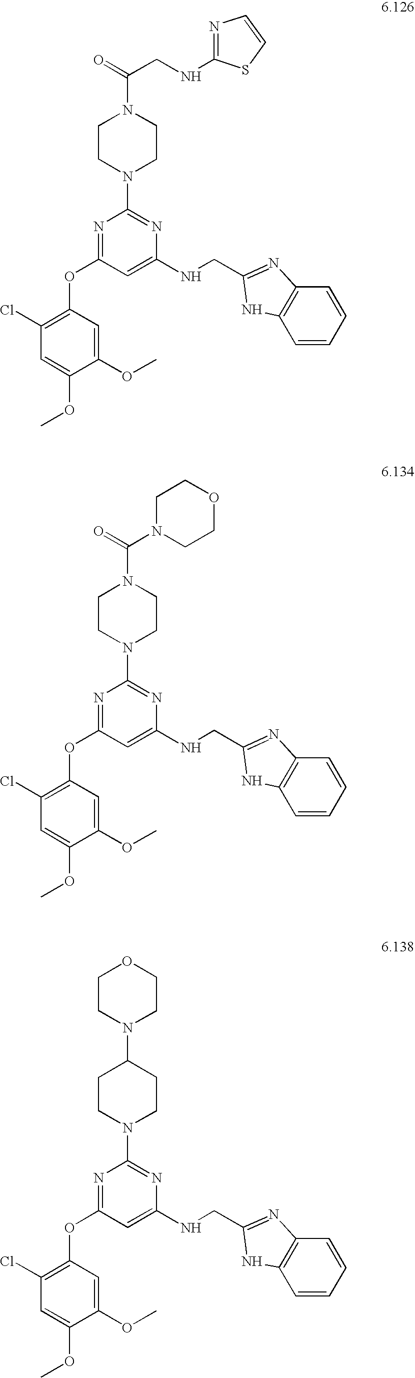

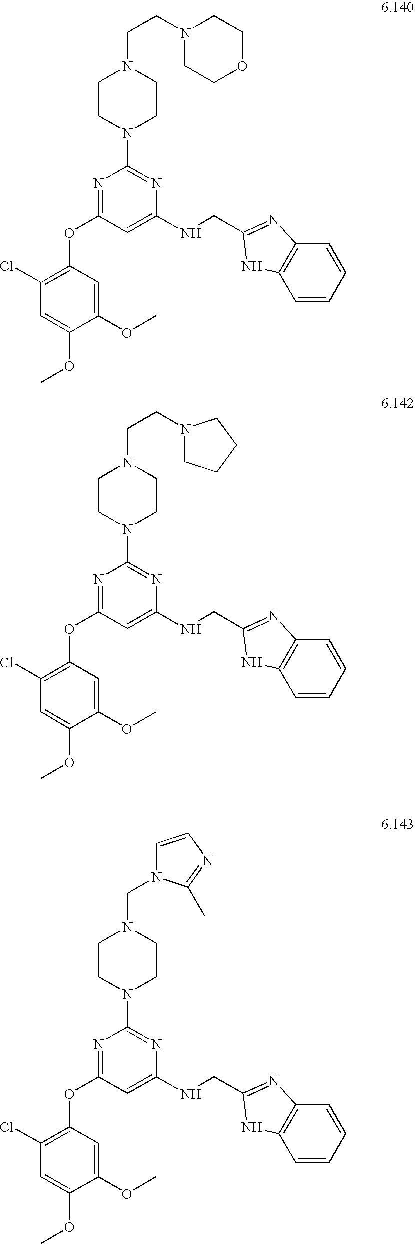

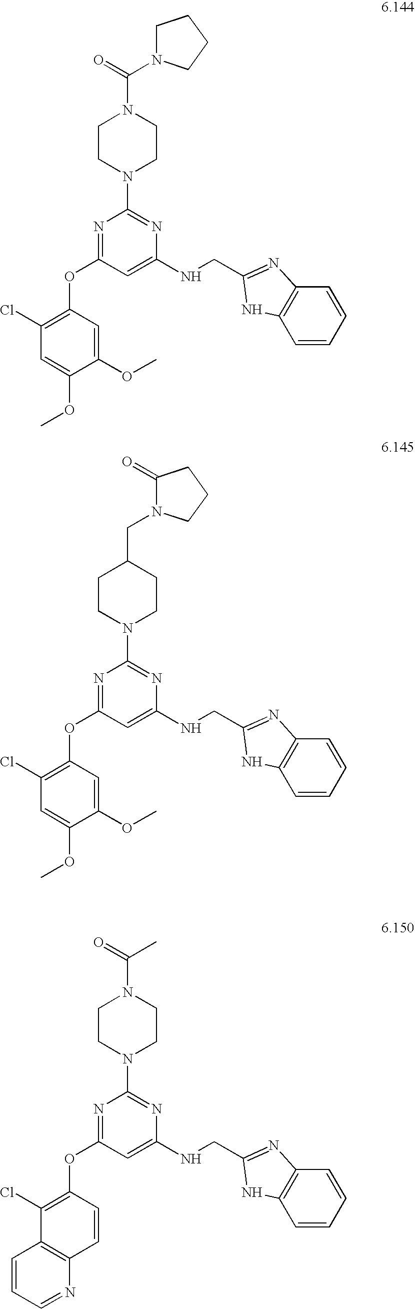

### Anitbacterial Activity Assays

Antibacterial activity as measured by the minimal inhibitory concentrations (MIC) and minimal bactericidal concentrations of compounds are well known (see., e.g., National Committee for Clinical Laboratory Standards 2000 Performance standards for antimicrobial disk susceptibility tests: approved standard, 7^{th} ed. M2-A7, vol. 20, no. 1, Committee for Clinical Laboratory Standards, Wayne, Pa.)

In vitro testing for antibacterial activity may be accomplished through use of a whole-cell bacterial growth inhibition assay. For example, an agar dilution assay identifies a substance that inhibits bacterial growth. Microtiter plates are prepared with serial dilutions of the test compound, adding to the preparation a given amount of growth substrate, and providing a preparation of bacteria. Inhibition of bacterial growth is determined, for example, by observing changes in optical densities of the bacterial cultures. Inhibition of bacterial growth is determined, for example, by comparing (in the presence and absence of a test compound) the rate of growth or the absolute growth of bacterial cells. Inhibition includes a reduction of one of the above measurements by at least 20%.

The compounds of the present invention are active against a wide range of bacteria. In preferred embodiments, the bacteria are Gram-positive bacteria including methicillin-susceptible and methicillin-resistant Staphylococci (including Staphylococcus aureus, Staphylococcus epidermidis, Staphylococcus haemolyticus, Staphylococcus hominis, Staphylococcus saprophyticus, and coagulase-negative Staphylococci), glycopeptide intermediary-susceptible Staphylococcus aureus (GISA), penicillin-susceptible and penicillin-resistant Streptococci (including Streptococcus pneumoniae, Streptococcus pyogenes, Streptococcus agalactiae, Streptococcus avium, Streptococcus bovis, Streptococcus lactis, Streplococcus sangius and Streptococci Group C, Streptococci Group G and viridans Streptococci), enterococci (including vancomycin-susceptible and vancomycin-resistant strains such as Enterococcus faecalis and Enterococcus faecium), Bacillus anthracis, Clostridium difficile, Clostridium clostridiiforme, Clostridium innocuum, Clostridium perfringens, Clostridium ramosum, Haemophilus influenzae, Listeria monocytogenes, Corynebacterium jeikeium, Bifidobacterium spp., Eubacterium aerqfaciens, Eubacterium lentum, Lactobacillus acidophilus, Lactobacillus casei, Lactobacilllus plantarum, Lactococcus spp., Leuconostoc spp., Pediococcus, Peptostreptococcus anaerobius, Peptostreptococcus asaccarolyticus, Peptostreptococcus magnus, Peptostreptococcus micros, Peptostreptococcus prevotii, Peptostreptococcus productus, Propionibacterium acnes, and Actinomyces spp. In more preferred embodiments, the Gram-positive bacterium is Staphylococcus aureus.

Table 3 lists the numbers of selected compounds with MIC values less than or equal to 8 μg/mL when tested against S. aureus.

TABLE 3
Compounds with MIC values ≦8 μg/mL against S. aureus.
Compound number

5.01, 5.05, 5.06, 5.07, 5.09, 5.10, 5.12, 5.17, 5.24, 5.26, 5.28, 5.30, 5.32, 5.35, 5.37, 5.50, 5.51,
5.53, 5.54, 5.55, 5.70
6.05, 6.06, 6.07, 6.12, 6.15, 6.19, 6.23, 6.29, 6.30, 6.43, 6.44, 6.45, 6.46, 6.47, 6.48, 6.49, 6.55,
6.56, 6.58, 6.59, 6.60, 6.61, 6.62, 6.63, 6.76, 6.83, 6.84, 6.85, 6.86, 6.87, 6.88, 6.89, 6.90, 6.91,
6.92, 6.93, 6.94, 6.95, 6.96, 6.97, 6.98, 6.99, 6.100, 6.101, 6.102, 6.103, 6.104, 6.105, 6.106,
6.107, 6.113, 6.119, 6.126, 6.130, 6.131, 6.133, 6.134, 6.135, 6.137, 6.138, 6.140, 6.141,
6.142, 6.143, 6.144, 6.145, 6.146, 6.147, 6.148, 6.149, 6.150

### MetRS Inhibition

Without being bound by theory, it is believed that the compounds of the present invention exert their antibacterial action through inhibiting MetRS.

The inhibition of MetRS activity may be monitored through use of in vitro bio-chemical assays through use of purified MetRS protein. Synthesis of a MetRS polypeptide can readily be accomplished through use of any of the various art-known techniques. For instance, a MetRS polypeptide can be synthesized chemically in vitro or recombinant DNA methods which are well known to those skilled in the art can be used to construct expression vectors containing MetRS coding sequences, and appropriate transcriptional/translational control signals. These methods include, for example, in vitro recombinant DNA techniques, synthetic techniques and in vivo recombination/genetic recombination. Alternatively, RNA capable of encoding target gene protein sequences can be chemically synthesized through use of synthesizers, for example.

The compounds of the present invention may also be evaluated for the ability to inhibit MetRS activity through use of known methods that measure the MetRS-dependent coupling of methionine to its cognate tRNA. In the assay, radiolabeled methionine, tRNA^{Met} and purified MetRS enzyme are incubated under appropriate conditions in the presence and absence of test compound and the amount of TCA-precipitable counts, which reflects the amount of methionine coupled to tRNA^{Met}, is determined. A titratable decrease in the amount of TCA-precipitable radioactivity in the presence of increasing compound indicates the test compound inhibits MetRS activity.

The following Table shows measured values for some selected compounds of the present invention.

TABLE 4
Examples of Formula 1 MetRS Inhibitors

| | S. Aureus MetRS |
|---|---|
| Examples # | IC₅₀ (μM) |
| 5.01, 5.05, 5.06, 5.07, 5.10, 5.11, 5.12, 5.16, 5.17, 5.18, 5.24, 5.25, | <50 nM |
| 5.26, 5.28, 5.29, 5.30, 5.31, 5.32, 5.34, 5.37, 5.50, 5.51, 5.53-5.56, | |
| 5.59, 5.65, 5.66, 5.67, 5.69, 5.70 | |
| 6.01, 6.05, 6.06, 6.07, 6.10, 6.12, 6.13, 6.14, 6.15, 6.19, 6.23, 6.29, | <50 nM |
| 6.30, 6.32, 6.34, 6.37, 6.39, 6.40, 6.41, 6.42, 6.43, 6.44, 6.45, 6.46, | |
| 6.47, 6.48, 6.49, 6.55, 6.56, 6.57, 6.58, 6.59, 6.60, 6.61, 6.62, 6.63, | |
| 6.77, 6.78, 6.83, 6.84, 6.85, 6.86, 6.87, 6.88, 6.89, 6.90, 6.91, 6.92, | |
| 6.93, 6.94, 6.95, 6.96, 6.97, 6.98, 6.99, 6.100, 6.101, 6.102, 6.103, | |
| 6.104, 6.105, 6.106, 6.107, 6.113, 6.119, 6.126, 6.130, 6.131, 6.133, | |
| 6.134, 6.135, 6.137, 6.138, 6.140, 6.141, 6.142, 6.143, 6.144, 6.145, | |
| 6.146, 6.147, 6.148, 6.149, 6.150 | |

Through well-known methods the inventive compounds and their pharmaceutically acceptable salts, solvates, esters and prodrugs may be formed into pharmaceutical compositions appropriate for the intended administration routes, such as for intravenous or intramuscular injection. Typically such compositions include various excipients, such as binders and buffers along with the pharmacologically-active compound.

The compounds and pharmaceutical compositions of the present invention are useful as antibacterial agents and, thus, may be used in methods to prevent or treat bacterial infections in animals. Treatment typically includes administering a pharmaceutically effective amount of a composition containing an antibacterial agent to a patient in need of such treatment, thereby inhibiting bacterial growth in the patient. Such a composition typically contains from about 0.1 to 90% by weight (such as 1 to 20% or 1 to 10%) of an anti-bacterial agent of the invention in a pharmaceutically acceptable carrier.

The efficacy of the present antibacterial compounds and pharmaceutical compositions in humans can be estimated in an animal model system well known to those of skill in the art (e.g., mouse and rabbit model systems of, for example, streptococcal pneumonia).

In a typical in vivo assay, an animal is infected with a pathogenic strain of bacterium, e.g., by inhalation of a bacterium such as Streptococcus pneumoniae, and conventional methods and criteria are used to diagnose the animal as being afflicted with a bacterial infection. The candidate antibacterial agent then is administered to the patient at a dosage of 1-100 mg/kg of body weight, or other suitable dosing regiment, and the animal is monitored for signs of amelioration of disease. Alternatively, the test compound can be administered to the animal prior to infecting the animal with the bacterium, and the ability of the treated animal to resist infection is measured. The results obtained in the presence of the test compound are compared with results in control animals that are not treated with the test compound. Administration of candidate antibacterial agents to the animal can be carried out through use of any route, such as oral, intravenous, topical, rectal, pulmonary.

The methods of the present invention prevent or treat a bacterial infection in a patient by administering a therapeutically effective amount of a compound of the invention. Typically a therapeutically effective amount should produce a serum concentration of active ingredient of from about 0.1 ng/mL to about 50-100 μg/mL. The pharmaceutical compositions typically should provide a dosage of from about 0.01 mg to about 2000 mg of compound per kilogram of body weight per day. The active ingredient may be administered at once, or may be divided into a number of smaller doses to be administered at intervals of time.

The precise dosing regimen and duration of treatment may be determined empirically and modified according to the professional judgment of the person providing treatment.

We claim:

1. A compound having the structure 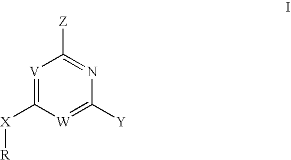
where X is O, S, N, or C,
exactly one of V and W is N, and the other is C—R₁,
R is optionally substituted aryl or heteroaryl,
R₁ is H, C₁₋₄ alkyl, or C₁₋₄ alkoxy,
Y is optionally substituted 1H-benzimidazol-2-ylmethyl, 1H-imidazol-2-ylmethyl or 2-(4-oxo-4,4a-dihydroquinolin-2-yl)hydrazine and
Z is an optionally substituted amino, thio, or heterocyclyl group,
as well as diastereomers, enantiomers or a pharmaceutically acceptable salt, solvates, esters and pro-drugs thereof.

2. The compound of claim 1, wherein X═O and R₁ is H.

3. The compound of claim 2, wherein R is a quinoline or a phenyl group bearing at least one substituent selected from the group consisting of halo, C₁₋₆ alkyl, C₁₋₆ alkoxy, trifluoromethyl, trifluoromethoxy, methylenedioxy, and cyano.

4. The compound of claim 3, wherein R is a phenyl group bearing at least one substituent selected from the group consisting of F, Cl, Me, OMe, CF₃, CF₃O, methylenedioxy, and cyano.

5. The compound of claim 4, wherein R is selected from the group consisting of 5-chloro-quinol-6-yl, 5-chloro-8-methoxy-quinol-6-yl, 2,4-dichlorophenyl, 2-chloro-4-methoxyphenyl, 3,4-dimethoxyphenyl, 2-chloro-4-fluorophenyl, 2-methyl-4-fluorophenyl, 2,4-difluorophenyl, 4-trifluoromethoxyphenyl, 3-methyl-4-chlorophenyl, 3,4-methylenedioxphenyl, 4-methoxyphenyl, 2-chloro-4-cyanophenyl, 2-chloro-4-trifluoromethylphenyl, 3,4,5-trimethoxyphenyl, 2,4-dichloro-3-methylphenyl, and 2-chloro4,5-dimethoxyphenyl groups.

6. The compound of claim 5, wherein R is selected from the group consisting of 5-chloro-quinol-6-yl, 5-chloro-8-methoxy-quinol-6-yl, 2,4-dichlorophenyl, 2-chloro-4-methoxyphenyl, and 2-chloro4,5-dimethoxyphenyl.

7. The compound of claim 1, wherein Z is selected from the group consisting of Z is NRₐR_{b}, where Rₐ and R_{b} are independently selected from the group consisting of H and lower alkyl, and the optionally substituted morphin-1-yl, thiomorphin-1-yl, piperazin-1-yl, pyrrolidin-1-yl, imidazol-1-yl, piperidin-1-yl, and 1,4-diazepan-1-yl.

8. The compound of claim 1, wherein Z is selected from the group consisting of optionally substituted morpholin-4-yl, dimethylamino, 4-acetylpiperazin-1-yl, 3,5-dimethylpiperazin-1-yl, 4-[2-(dimethylamino)ethyl]piperazin-1-yl, 3-hydroxypyrrolidin-1-yl, 3-oxopiperazin-1-yl, 4-(furan-2-ylcarbonyl)piperazin-1-yl, 4-methyl-1H-imidazol-1-yl, 4-morpholin-4-ylpiperazin-1-yl, 4-acetyl-1,4-diazepan-1-yl, 4-pyrimidin-2-ylpiperazin-1-yl, 4-(cyclopropylcarbonyl)piperazin-1-yl, 1,1-dioxidothiomorpholin-4-yl, 4-pyridin-2-ylpiperazin-1-yl, 3-[acetyl(ethyl)amino]pyrrolidin-1-yl, 4-(2-methylpropanoyl)piperazin-1-yl, 4-[5-(trifluoromethyl)pyridin-2-yl]piperazin-1-yl, 4-pyrazin-2-ylpiperazin-1-yl, 4-(2-morpholin-4-ylethyl)piperazin-1-yl, 4-pyridin-4-ylpiperazin-1-yl, 4-(4-methoxypyrimidin-2-yl)piperazin-1-yl, 3-oxo-1,4-diazepan-1-yl, 4-(1,3,5-triazin-2-yl)piperazin-1-yl, 4-(1,3-thiazol-2-yl)piperazin-1-yl, 4-[2-(1H-imidazol-1-yl)ethyl]piperazin-1-yl, 4-[(1,3-thiazol-2-ylamino)acetyl]piperazin-1-yl, 4-(morpholin-4-ylcarbonyl)piperazin-1-yl, 4-(pyrrolidin-1-ylcarbonyl)piperazin-1-yl, 4-[2-(2-methyl-1H-imidazol-1-yl)ethyl]piperazin-1-yl, 4-(2-pyrrolidin-1-ylethyl)piperazin-1-yl, or 4-[(2-oxopyrrolidin-1-yl)methyl]piperidin-1-yl.

9. The compound of claim 8, wherein Z is selected from the group consisting of optionally substituted 4-acetylpiperazin-1-yl, 3-oxopiperazin-1-yl, dimethylamino, 4-pyrimidin-2-ylpiperazin-1-yl, 3,5-dimethylpiperazin-1-yl, 4-(cyclopropylcarbonyl)piperazin-1-yl, 4-pyridin-2-ylpiperazin-1-yl, 4-(2-methylpropanoyl)piperazin-1-yl, 4-(2-morpholin-4-ylethyl)piperazin-1-yl.

10. The compound of claim 1 wherein V═CH and W═N.

11. The compound of claim 10, selected from the group consisting of

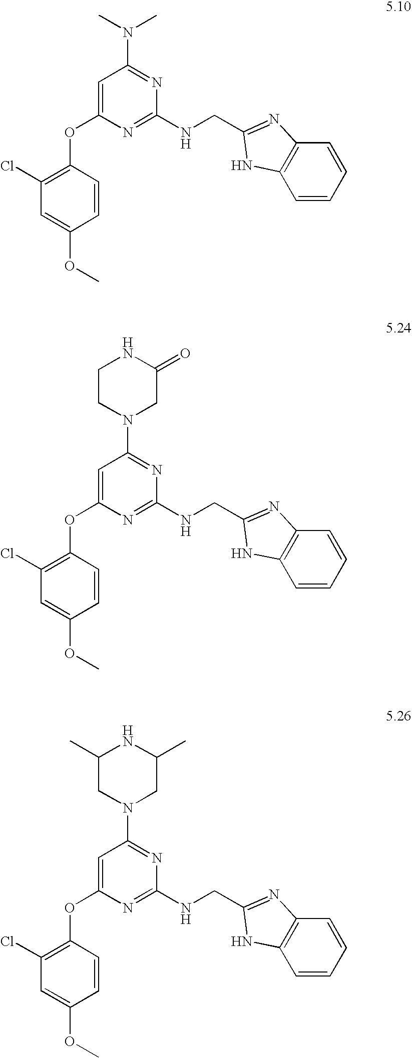

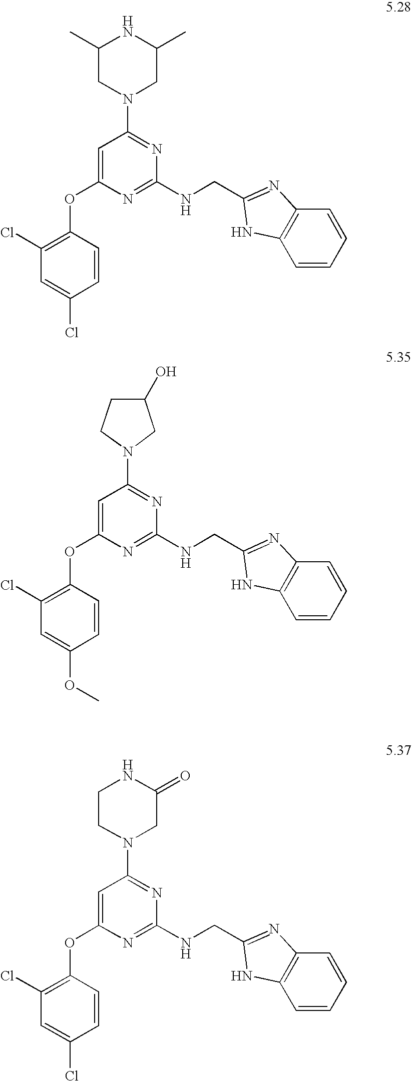

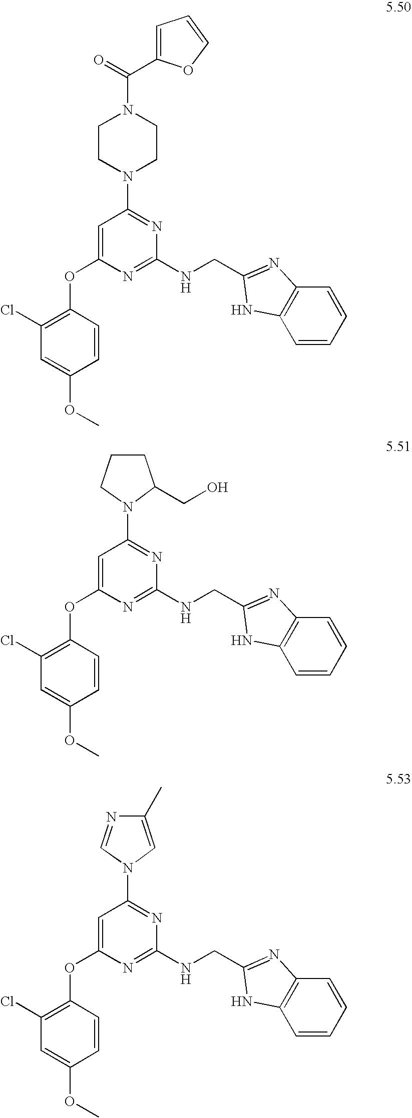

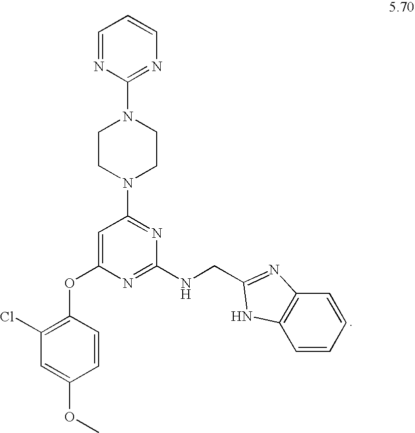

12. The compound of claim 1, wherein V═N and W═CH.

13. The compound of claim 12, selected from the group consisting of

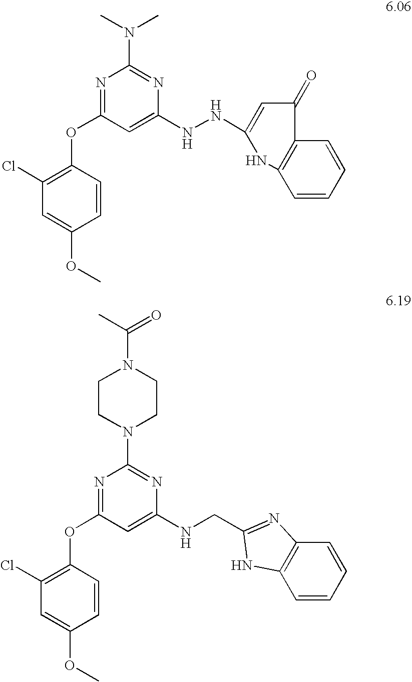

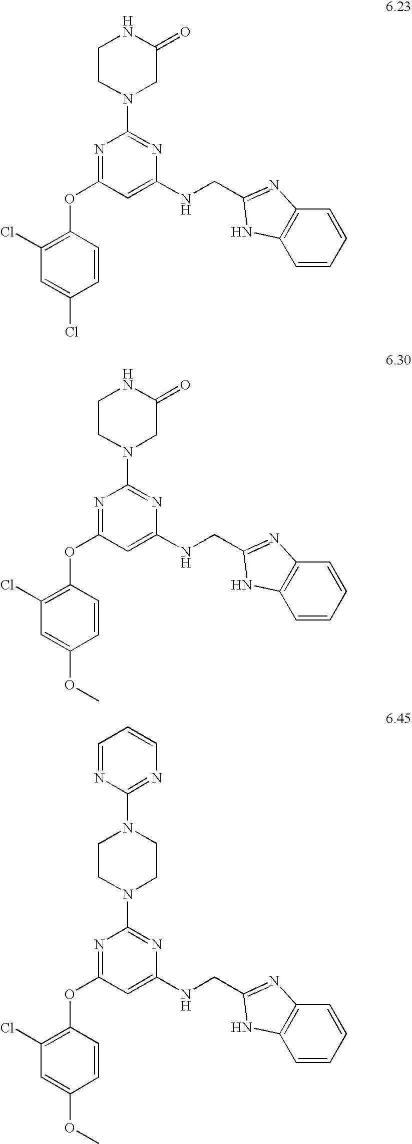

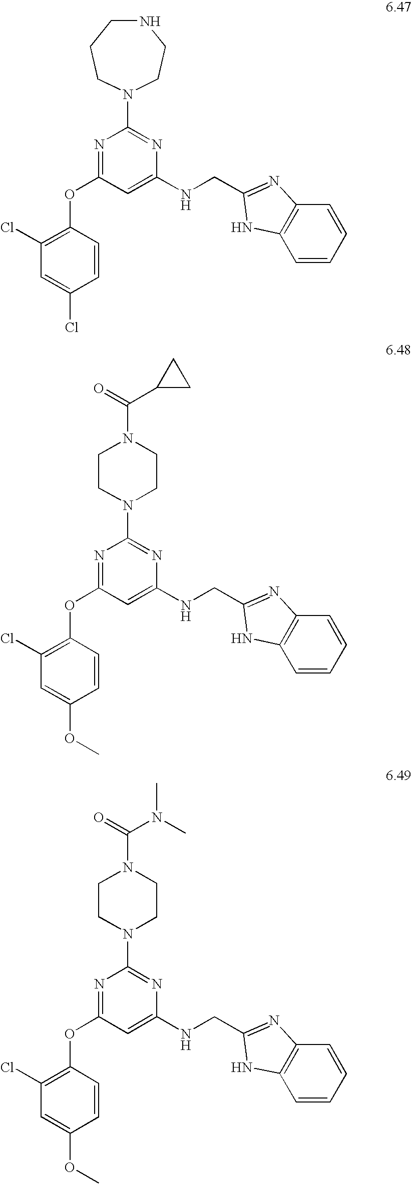

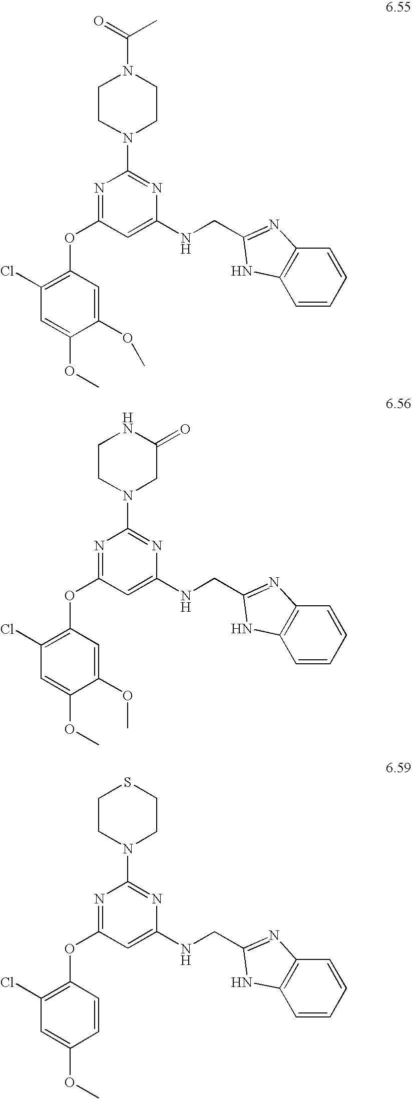

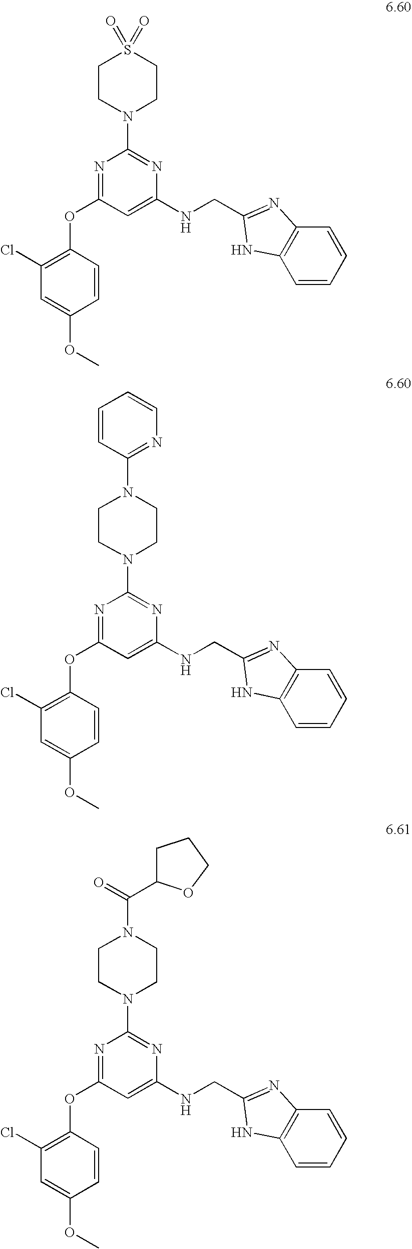

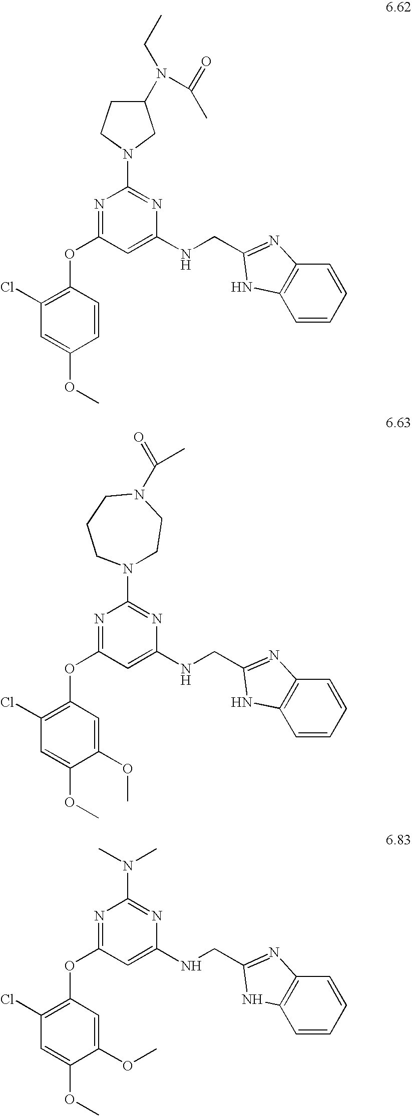

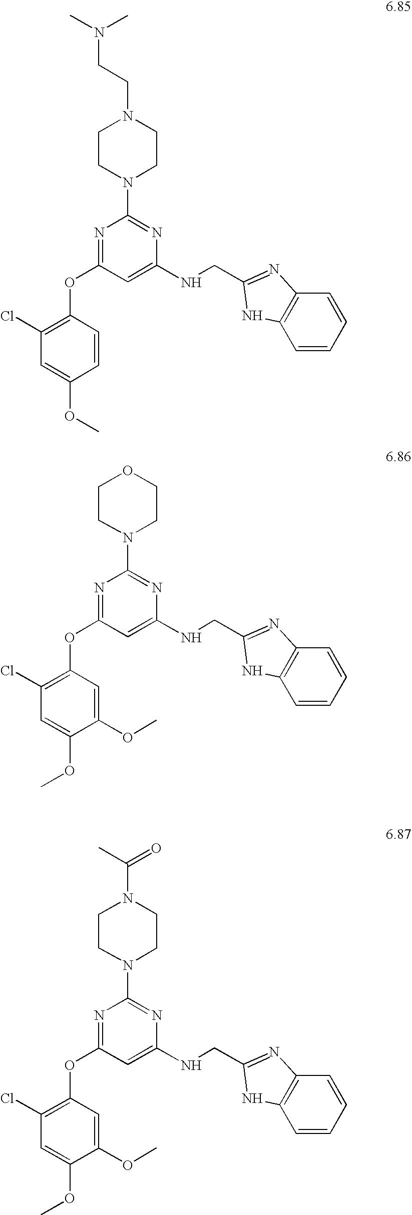

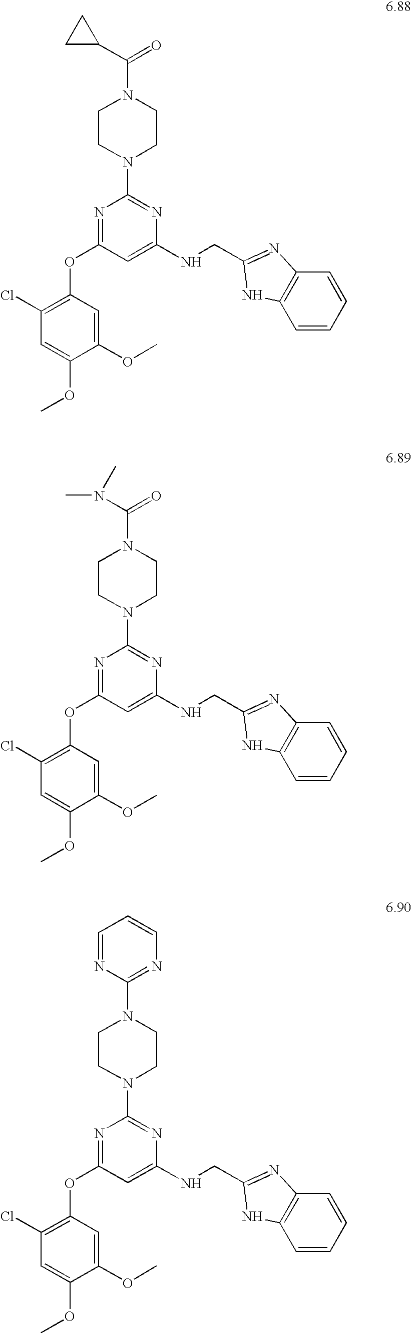

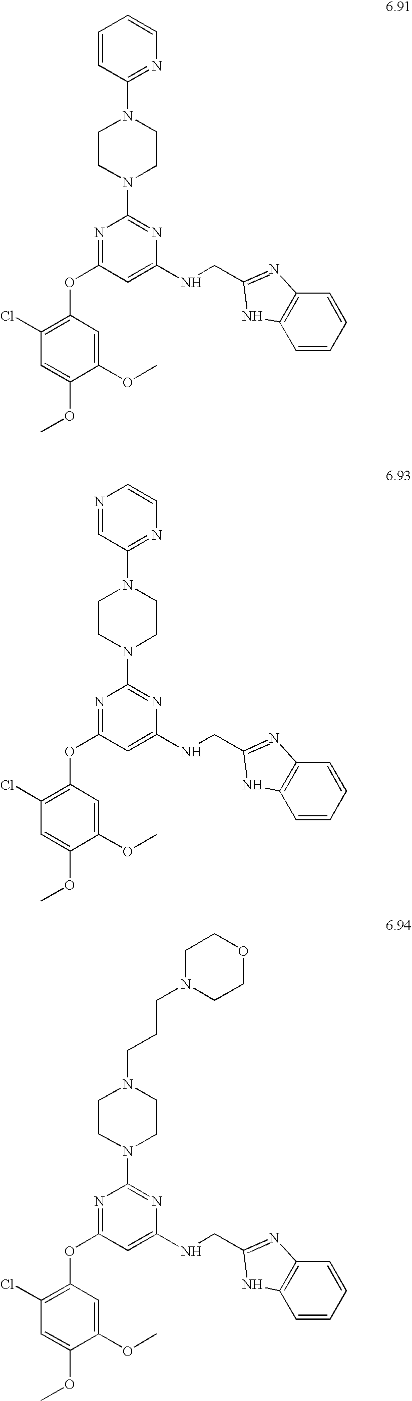

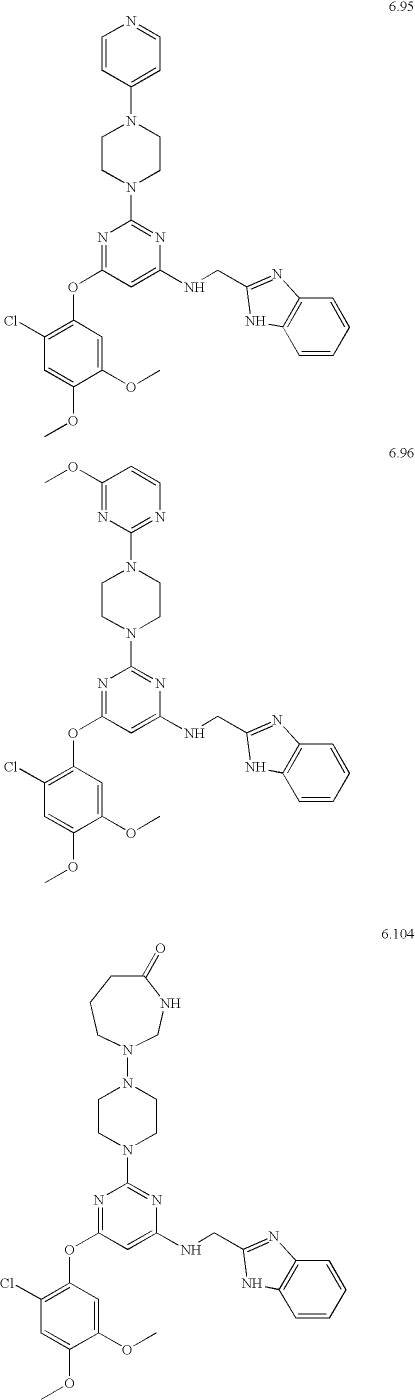

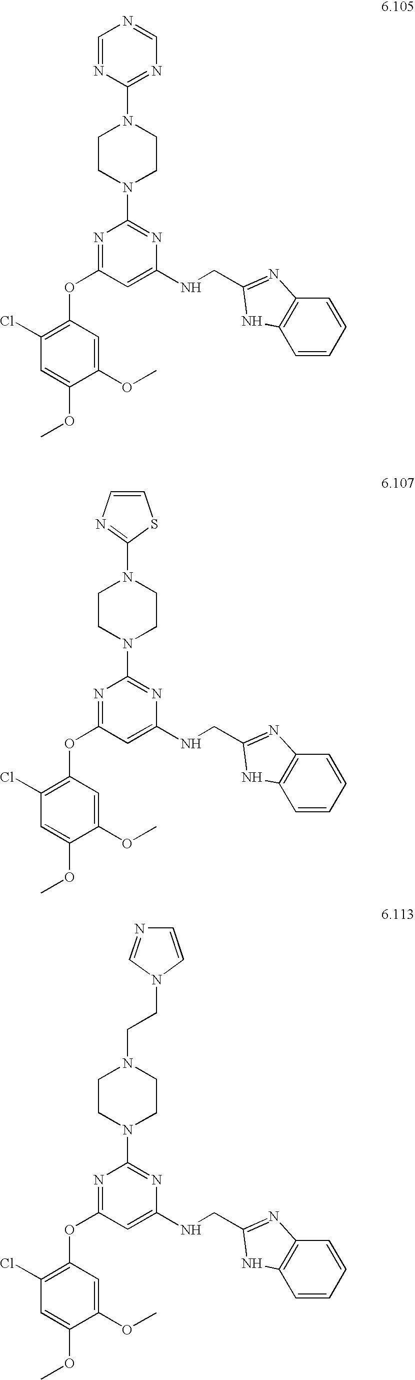

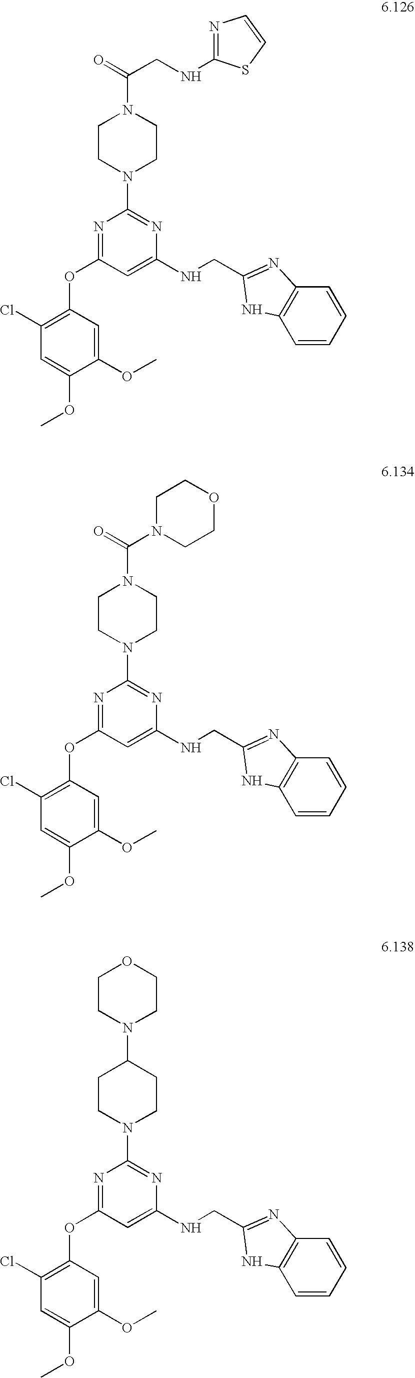

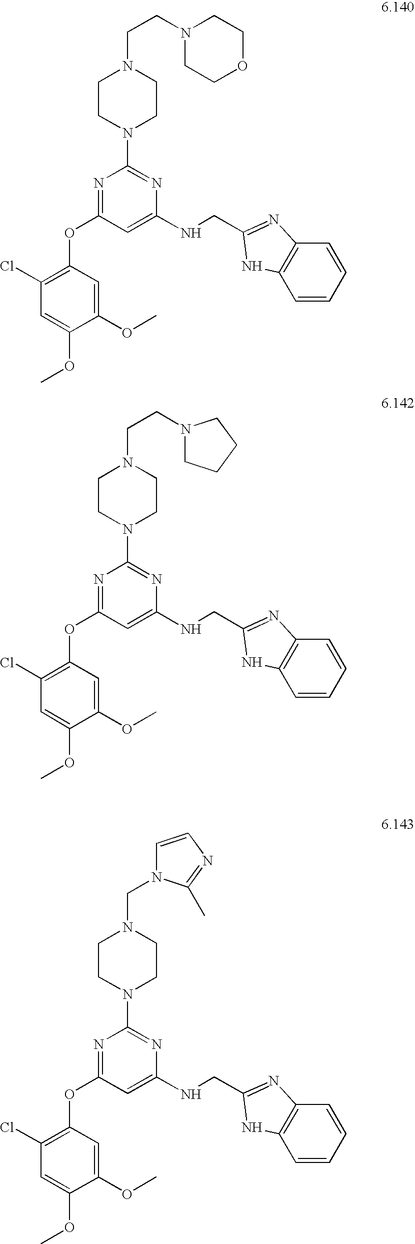

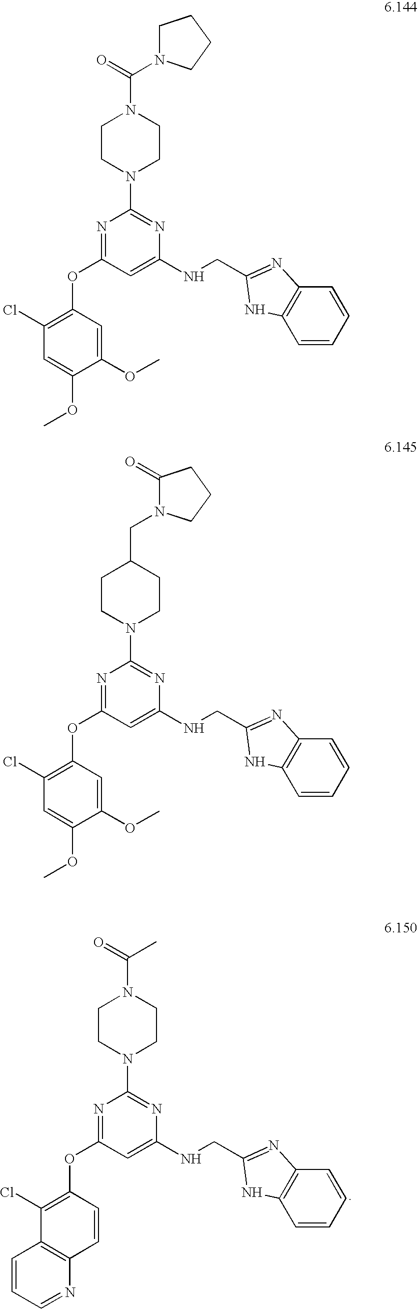

14. A pharmaceutical composition, comprising a compound of claim 1 and a pharmaceutically acceptable diluent or carrier.

15. A method of making a compound of claim 1, comprising the step of treating a compound of formula I 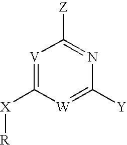
where
x═O,
R is optionally substituted phenyl or quinol-6-yl,
exactly one of V and W is N, and the other is CH,
and Y and Z are both selected from the group consisting of halo, alkylthio, alkylsulfonate, alkylsulfinate and optionally substituted 1H-benzimidazol-2-ylmethyl

16. A method for treating a bacterial infection in a patient, comprising administering to the patient an effective amount of a pharmaceutical composition of claim 14.

17. The method of claim 16, wherein the bacterium is Gram-positive.

18. The method of claim 17, wherein the Gram-positive bacterium is selected from the group consisting of Staphylococcus, Streptococcus, Enterococcus, Clostridium, Haemophilus, and Listeria spp.

19. The method of claim 18, wherein the bacterium is Staphylococcus aureus.

20. The compound of claim 16, wherein the compound is administered at a dosage between about 1 and 1000 mg/kg.

21. The compound of claim 20, wherein the dosage is between about 100 and 1000 mg/kg.

22. The compound of claim 21, wherein the dosage is between about 10 and 100 mg/kg.

23. The method of claim 16, wherein the pharmaceutical composition is administered by a route selected from the group consisting of intravenous, oral, rectal, intramuscular, subcutaneous, and pulmonary administration.
